(19) United States
(12) Patent Application Publication
(10) Pub. No.: US 2022/0160255 A1
(43) Pub. Date: May 26, 2022

(54) DEVICE, PROCESS AND COMPUTER PROGRAM FOR INFLUENCING THE BREATHING OF A PERSON

(71) Applicant: Drägerwerk AG & Co. KGaA, Lübeck (DE)

(72) Inventors: Marcus EGER, Lübeck (DE); Lorenz KAHL, Lübeck (DE); Philipp ROSTALSKI, Lübeck (DE); Eike PETERSEN, Lübeck (DE)

(21) Appl. No.: 17/440,602

(22) PCT Filed: Mar. 20, 2020
(86) PCT No.: PCT/EP2020/057717

§ 371 Date: Sep. 17, 2021

(30) Foreign Application Priority Data
Mar. 20, 2019 (DE) 10 2019 001 926.1

(51) Int. Cl. A61B 5/087; A61B 5/389; A61M 16/00

(52) U.S. Cl. CPC A61B 5/087; A61B 5/389; A61M 16/026; A61M 16/0051; A61M 2016/0042; A61M 2205/582; A61M 2205/583; A61M 2016/0039; A61M 2205/581

(57) ABSTRACT

A device, a process and a computer program influence the breathing of a person. The device (10) for influencing the inspiratory muscles of a person (20) includes a detection device (12) for detecting an electromyographic signal of the person; a breathing influencing device (14) and a control device (16) for controlling the detection device (12) and the breathing influencing device (14). The control device (16) is configured to determine information on a muscle state of an inspiratory muscle of the person (20) on the basis of the electromyographic signal. The control device (16) is further configured to operate the breathing influencing device (14) as a function of the information on the muscle state in a training mode, which is limited in time, with a training intensity.

### CROSS REFERENCE TO RELATED APPLICATIONS

This application is a United States National Phase Application of International Application PCT/EP2020/057717, filed Mar. 20, 2020, and claims the benefit of priority under 35 U.S.C. § 119 of German Application 10 2019 001 926.1, filed Mar. 20, 2019, the entire contents of which are incorporated herein by reference.

### TECHNICAL FIELD

Exemplary embodiments pertain to a device, to a process and to a computer program for influencing the inspiratory muscles of a person, especially but not exclusively to a concept for training the inspiratory muscles of a training person or of a patient based on electromyographic signals of his/her inspiratory muscles.

### TECHNICAL BACKGROUND

Various concepts which bring about a training of the inspiratory muscles are known in the conventional technique. These are based, as a rule, on a visualization of a breathing performance, which exceeds the usual breathing performance. The training person is thus stimulated to perform an intensified breathing, which strengthens the inspiratory muscles beyond the normal use. Further, it is known that breathing can be made difficult, which is achieved, for example, by reduced cross sections of mouthpieces or the like. The difficulty of breathing can be set in this connection, as a rule, so that difficulties ranging from a low level of difficulty of breathing to gradually increasing resistances to breathing can be set in increments. Such a type of inspiratory muscle training is known for both increasing the performance capacity during sports activities and for supporting recovery and for rehabilitation after illnesses in which the inspiratory muscles were weakened or affected.

### Types of Inspiratory Muscle Training (IMT)

Different training modalities/modes are known, in principle, for carrying out an inspiratory muscle training. The different training modalities differ, in part, in the manner of the pneumatic intervention. Moreover, the different training modalities also have at times different therapeutic goals. While some training modalities have rather the task of improving the muscle strength, other training modalities have, in turn, rather the goal of improving the endurance (muscle endurance). Some training modalities are said in medical research to serve both goals. A review of different training modes/modalities can be found in the book “Textbook of Pulmonary Rehabilitation” in the Chapter “Inspiratory Muscle Training” by Daniel Langer. Especially Sections 18.3.4 through 18.3.7 and Table 18.3 are of interest in this connection.

Aside from the special case of normocapnic hyperpnea, these methods have in common that a passive resistance element is introduced into the airway. This element is partly regulated/adjustable. In a further sense, these also include a flow limitation, occlusion as well as pressure limitation. Passive means in this case that the inspiratory muscle training device does not generate a pressure and/or a volume flow itself. The inspiratory muscle training device would be a passive element (resistance, potentiometer, the resistance possibly having especially nonlinear voltage/current characteristics, or it is regulated). However, it is not an active voltage or current source (no energy is introduced). Various devices are available commercially for this type of inspiratory muscle training with a pneumatic resistance. These are at times mobile (hand-held) devices, which the patient/the training person holds, themself, in front of their mouth in order to breathe through it.

Moreover, the detection and recording of the performance capacity of the inspiratory muscles are known, especially for the field of medicine. If, for example, a patient is ventilated mechanically, the mechanical work performed by the inspiratory muscles is taken over at least partially by a ventilator. The inspiratory muscles may be damaged due to the sustained inactivity already after a few hours. Thus, often it is not easy to change a patient over again to an independent breathing after a prolonged mechanical ventilation. Even if the illness of the patient, which originally led to the ventilation, is not present any longer, it is often impossible to simply disconnect the patient from the ventilator. The inspiratory muscles atrophied during the inactivity are now no longer able to perform the work of breathing necessary for the patient who is at rest, i.e., is lying inactively in the bed.

US 2009 0 229 611 A1 describes different approaches to how an inspiratory muscle training can be carried out for patients being ventilated. It is described there how such an inspiratory muscle training can be integrated into a ventilator. According to that document, one shortcoming of prior-art processes for inspiratory muscle training is that the patient is required to make a defined effort only at the beginning of each breath. Pneumatic interventions in the mechanical ventilation are therefore presented, in which it is ensured that the patient must make a defined work of breathing over the entire breath.

Contrary to the above-described devices for inspiratory muscle training, a ventilator frequently offers additional and partially different possibilities for pneumatic intervention. It is decisive in this connection that the ventilator represents an active pressure/volume flow source. On the one hand, it is possible to simulate a corresponding pneumatic resistance against the patient by a corresponding pressure or volume regulation. Moreover, other possibilities of stressing the inspiratory muscles are available as well due to the active pressure/flow source. One example would be, for example, the regulation of the breathing performance performed by the patient themself to a constant value. The ventilator would then bring up the respective part going beyond this actively or introduce corresponding obstructions/difficulties/resistances (if the breathing performance needed is actually much lower, as is necessary for training purposes). For further details of a ventilator, we are referring to the Preliminary Published Patent Application DE 10 2015 011 390 A1 and especially to FIG. 4 and to the corresponding description (DE 10 2015 011 390 A1 is incorporated herein by reference).

Another peculiarity of the inspiratory muscle training in connection with ventilated patients is also the fact that the inspiratory muscle stressing is brought about in part/as a rule by a lower relief. A ventilated patient is not, as a rule, able to perform the necessary work of breathing themself, not even the work of breathing necessary for the physical state of rest. He/she is therefore connected to a ventilator. The ventilator takes over the work of breathing of the patient completely or at least partially and relieves him/her thereby. An inspiratory muscle training can already be achieved in this setting by the relief being set or reduced temporarily. A lower relief thus effectively already represents a stressing of the inspiratory muscles.

In addition, impairments resulting from the ongoing ventilation are to be taken into consideration in a patient being ventilated. These include, among other things, maximum pressures, minimum volumes, etc. In addition, alarm limits my possibly have to be adjusted during the inspiratory muscle training.

The Preliminary Published Patent Application “Device, Process and Computer Program for Ventilating a Patient,” DE 10 2015 011 390 A1, describes a process in which the assist by the ventilator is adaptively adapted to the state of fatigue of the patient, which is determined by EMG (electromyography). The goal of the regulation is to maintain the fatigue index of the patient, which is derived from sEMG (surface electromyogram, surface EMG) at a constant value. The assist by the ventilator is increased in case of a rising fatigue index (higher degree of fatigue). The work of breathing to be provided by the patient decreases and the inspiratory muscles can recover. In case of a declining fatigue index, the assist by the ventilator is reduced, so that a larger part of the work of breathing must be performed by the patient themself.

“Surface Electromyography: Physiology, Engineering and Applications” by Roberto Merletti and Dario Farina (2016, ISBN-13:978-1118987025) describes a plurality of possible applications of surface EMG in rehabilitation and training. For example, the monitoring of the degree of muscular exhaustion, of the contribution of different muscle groups to a movement as well as of training-induced changes of the state of the muscles shall be mentioned. Another prominent field of application are biofeedback methods, in which the visual representation of EMG-based activation indicators for the patient are used to control and to optimize the training.

The web site “insight Instruments—What is Biofeedback?,” URL: https://web.archive.org/web/20180409201831/http://www.biofeedback.co.at/alles-ueber-biofeedback/was-ist-biofeedback, published on Apr. 9, 2018, offers an introduction to biofeedback. It comprises the basic functions of biofeedback as well as various applications as well as its cost, advantages and risks. It describes the application of EMG (surface electromyography, surface EMG) for certain muscle tensions.

The design and control of inspiratory muscle training based solely on respiratory parameters, which depend essentially on the overall mechanical performance capacity of the inspiratory muscles, fails to take into consideration any detailed information on the state of the inspiratory muscles to be trained. This applies to both the adaption of the training regimen and the analysis of the training performance of the person or of the patient.

Due to the fact that only a very limited amount of information is available in the state of the art on the state of the muscles, the existing methods are only adaptive to a low extent as well. This low level of adaptivity pertains both to the possibility of adapting the training load within a training session, in which the training mode is used, and to adaptations for the subsequent training sessions. Neither of them can be optimally adapted to the degree of exhaustion or recovery of the muscles; overtraining or undertraining with serious consequences especially for persons or patients on training devices and especially on ventilators are possible. Optimal training is important for a good weaning score. Good weaning is, in turn, fundamental for a good clinical result.

The interaction between the training person or patient and the training device (for example, a ventilator) is not currently optimal from two points of view. On the one hand, the training person/patient receives no or little feedback during a training on his current performance in respect to the target performance, which may lead to lack of motivation and hence to reduced training success and to a less coordinated effort and hence, for example, to incorrect stresses, for example, to an undesirably strong expiratory activity. On the other hand, it is not currently possible especially for the patient to determine themself the time and the degree of difficulty of the training session; it is thus determined externally, which may likewise have strong negative consequences on the motivation of the patient.

### SUMMARY

There is correspondingly a need to detect the degree of exhaustion or the degree of recovery of the muscles during and after the training more accurately and to take this into consideration for planning and monitoring the training phases and for visualizing and analyzing the training results. This need is met by exemplary embodiments of a device, breathing unit and process according to the invention.

An inspiratory muscle training to be carried out with the use of an inspiratory muscle training device or ventilator is therefore provided. Information on the state of a muscle is generated on the basis of EMG measurements. It is assumed in this connection, on the one hand, that a ventilator is used as the training device. On the other hand, an EMG-based muscle state monitoring unit is also carried out with usual inspiratory muscle training devices (for example, hand-held devices, which are also suitable for home use).

EMG measurements of the inspiratory muscles are optionally combined with pneumatic signals in order to make possible a comprehensive monitoring of the state of the inspiratory muscles, as a result of which the inspiratory muscle training can be better adapted to the patient or person individually and monitored. These pneumatic signals comprise:
Pressures, volume flows (flows) and variables derived therefrom, e.g., volumes or time products (e.g., pressure time product). In particular, the Paw (airway pressure at the patient port), PEEP (positive end-expiratory pressure), Pᵢₙₛₚ (inhalation pressure), patient flow, tidal volume, minute volume, pneumatic lung resistances, lung elasticity, breathing performance, time constants of breathing.
Gas properties, such as concentrations, partial pressures, fractions or temperatures, especially oxygen O₂, carbon dioxide CO₂, water H₂O. The end-tidal CO₂ (etCO₂) shall also be mentioned in the case of capnographic signals (CO₂). Substances (nebulized drugs, anesthetics, helium) that are introduced into the breathing gas.
Properties of the lungs derived from pressure and/or volume flows (flow) and gas properties, e.g., lung volumes.

The inclusion of EMG before, during and between the individual training sessions makes it possible to take into consideration more accurate information on the state of the muscles to be trained. As a result, the course of the training can be better adapted to the individual needs of the person or patient. Moreover, the feedback of the information obtained from the EMG to the training person/patient makes possible an improved interaction with the training device.

Exemplary embodiments of the present invention are correspondingly based on the core idea of an improved detection of the muscle state of the inspiratory muscles of a person or of a patient, who is being ventilated or is training. The detected muscle state is used to plan and to control trainings to strengthen the inspiratory muscles, which degenerate during ventilation, or lead to increased performance capacity of the inspiratory muscles. More accurate detection of the muscle states, which allow a refined parameterization and monitoring of the training, is possible due to the use of electromyography (EMG).

How the weaning from mechanical ventilation can be carried out in the most successful manner possible is the subject of current research. An action proposed in this connection is the training of the inspiratory muscles by inspiratory muscle training (IMT). The inspiratory muscles of the patient are challenged by training sessions with a temporarily increased load during inspiratory muscle training. These training sessions shall improve the strength and/or the endurance of the inspiratory muscles again to the extent that the patient can breathe again permanently without assist.

Since long-term overstraining of the inspiratory muscles is also harmful for the patient or for the training person, in addition to chronic underchallenge of these muscles, monitoring of the activity as well as of the general state of the inspiratory muscles has increasingly shifted of late into the focus of clinical research.

The requirements imposed on an inspiratory muscle training are, in general,
The training should be as effective as possible in order to make independent breathing possible for the person/patient as quickly as possible.
The inspiratory muscle training should be as sustained as possible. The patient should be prevented from experiencing a relapse and from having to be connected again to a ventilator.
The inspiratory muscle training should be as gentle as possible for the patient.
The expense to the hospital should be kept at a relatively low level.

These requirements can also be applied in the narrower sense of the word to rehabilitation and/or to the weaning of patients from ventilation devices or, in a broader sense, also to the training of the inspiratory muscles of persons, especially athletes.

Various, therapeutically relevant observations can be made on the state of the inspiratory muscles on the basis of EMG alone as well as possibly in combination with pneumatic data and information on the physiology of the respiratory system. To obtain comprehensive information on the state of the breathing-relevant muscles, it is helpful to analyze a plurality of EMG leads rather than analyzing only one EMG lead. In addition to the detection of the diaphragmatic activity, these also include EMG measurements of the intercostal muscles, of additional accessory inspiratory muscles as well as of the antagonists, for example, the abdominal muscles, and the rectus abdominis muscle.

1. Amplitude

For example, the following clinically relevant information can be determined on the basis of the EMG amplitude:
A physiologically non-meaningful ratio of the activity of intercostal and diaphragmatic muscles or generally the ratio of the different muscles involved in breathing. This distribution of the muscles can also be determined separately for the phase of inhalation and for the phase of exhalation.
strong expiratory activity,
anticyclic activity (asynchrony of the patient and the ventilator),
generally: the time curve of the neuronal activation of the inspiratory muscles within one breath,
thoracic strain without breathing, for example, on rising up of the upper body. Both the diaphragm, the intercostal muscles and the expiratory muscles are active now. These may be, for example, changes in posture.

2. Mechanical Stress

For example, the following information can be obtained on the mechanical stress of the respiratory system in combination with pneumatic measured values:
The time curve of the breathing performance performed by the person or the patient as well as the time curve of the pressure applied by the patient.
The time curve of the breathing performance pertaining to the respiratory system (provided by the person/patient and the training system together).
The maximum pressure difference dropping over the lungs. This also includes the maximum force and the maximum pressure.
Taking into account the influence of antagonistic activity on the inspiratory muscles (for example, abdominal muscles, rectus abdominis muscle).

The pressure difference dropping over the lungs or also the pressure difference that “opens” the lungs is also called driving pressure. If the driving pressure is too low, the lungs will not be sufficiently recruited. Collapse of alveoli will occur. If the driving pressure is too high, pulmonary tissue will possibly be hyperdistended and thus damaged.

3. Unusual Contractions

It is further possible by means of different signal processing methods to detect unusual muscle contractions, for example, spasms or cough or hiccups on the basis of EMG signals.

4. Fatigue

In addition, the state of fatigue of the inspiratory muscles (muscle fatigue) can be detected from the EMG. Muscle fatigue is a state in which the muscle has only limited ability to generate or to maintain a force. A developing muscle fatigue can be detected from changes in the EMG signal already before the time at which the muscles are no longer able to perform a task. Various methods are known to this end for calculating a fatigue index, which describes the degree of beginning fatigue from the EMG signal. Various methods for calculating an EMG-based fatigue index are known. Muscle fatigue can also be studied especially as a function of the time both within one session or over a plurality of sessions.

5. “Internal” States

Finally, different pathological muscle states can be detected based on EMG on the basis of signal classification algorithms. Pathological states of the entire respiratory system (for example, Cheyne Stokes breathing) can be detected on the basis of EMG.

Additional EMG-based diagnostic methods are neuromuscular efficiency as well as muscle regeneration. The muscle regeneration may be quantified, for example, by DOMS (Delayed Onset Muscle Soreness). Muscle regeneration is also important especially in planning the time for the next training. Other methods are electromuscular delay as well as the detection of the risk of atrophy.

6. Additional Information that is Also Generated in Connection with the EMG Signal

Cardiogenic EKG signal components, which can be extracted by signal processing, are also present in the respiratory EMG signal. The information in these signal components can likewise be analyzed and used. This applies, for example, to the stress affecting the entire body. For example, the heart rate can be determined for this. Especially the heart rate variability (HRV) is suitable for quantifying the stress level.

Moreover, it is also possible to calculate a stress level from the transition impedance of the electrodes. The production of sweat, which is connected with increased stress, leads to changed electrical effects at the electrodes and can be detected.

Different methods, with which EMG-based information can be determined on the state of the inspiratory muscles, were described in the above section. In order to make it possible to use this information in a meaningful manner in the course of an inspiratory muscle training, this information must be evaluated, weighted and summarized. The individual state information is processed in inspiratory muscle training such that adaptation recommendations can be generated from them for the inspiratory muscle training.

The state processing takes place in two parallel units or modules:
Possible adaptation steps, which immediately affect the ongoing training session, are determined within the framework of the on-line analysis during an ongoing training session.
Each training unit is analyzed retroactively within the framework of the off-line analysis. The information obtained now can be used by a planning unit for planning the next training session.

Both units solve, in essence, an optimization task with secondary conditions: By adapting the intensity of training as well as the training mode, the benefit of the training shall be maximized, doing so under the secondary condition that no states of the muscles or of the respiratory system that are hazardous for health shall be reached. The result is always a recommendation for the selection of the training intensity in the next time unit, wherein the history of the training shall, in particular, be taken into consideration as well. The effect certain training intensity levels or changes in the training intensity had on the person or on the patient in the past can, in particular, be taken into consideration here. This recommendation is directed towards the training control in the on-line training unit and it shall immediately affect the ongoing training session. In case of the off-line analysis, the recommendation is directed towards the planning unit and shall be included by this in the planning of the next training session.

Exemplary embodiments of the present invention create a device for automatically influencing the inspiratory muscles of a person, with a detection device for detecting an electromyographic signal of the person with a breathing influencing device; and with a control unit for controlling the detection device and the breathing influencing device. The control device is configured to determine information on a muscle state of an inspiratory muscle of the person on the basis of the electromyographic signal. The control device is further configured to operate the breathing influencing device as a function of the information on the muscle state in a training mode limited over time with a training intensity. The training mode can thus advantageously be planned taking the muscle state into consideration and also corrected during the performance.

In some other exemplary embodiments, the device for influencing the inspiratory muscles of a person may be configured as an inspiratory muscle training device, which may be configured as a mobile inspiratory muscle training device in exemplary embodiments. An improved handling of the device, which also allows a mobile use independently from supply networks, and which can be operated, for example, independently from the power grid or in the home, can thus advantageously be achieved.

In other exemplary embodiments, the control device may be configured, furthermore, automatically to carry out the training mode as a difficulty of the breathing of the person for training the inspiratory muscles. As a result, a systematic training can advantageously be carried out, during which especially the duration and/or the intensity and/or the level of difficulty can be increased, for example, continuously.

The control device may be configured in some other exemplary embodiments automatically to adapt the influencing of the breathing of the person adaptively by the breathing influencing device as a function of the information on the muscle state. Both an imminent overstraining as well as a needless underchallenge of the muscles being trained can advantageously be reduced or completely avoided by the adaptation of the training mode.

The control device may further be configured in exemplary embodiments, for example, to automatically influence the training mode in terms of a training duration or the intensity/level of difficulty. The stress on the muscles being trained can advantageously be influenced by means of these parameters especially efficiently.

The control device may be configured in some exemplary embodiments to stop or automatically interrupt the training mode as a function of the information on the muscle state. Overstraining of the muscles being trained can thus advantageously be avoided. Such an overstraining may possibly lead to permanent damage to the muscles.

In some exemplary embodiments, the stopping or the interruption of the training mode can be carried out by the control device automatically as a function of a signal. The signal may indicate at least one piece of information on at least one element of the group comprising an expiratory activity, an inspiratory activity, of harmful pressure conditions in the lungs, of sustained spasms, cough or hiccup, muscle fatigue above a threshold considered to be intolerable and detection of pathological states, which may be harmful to a person. A disadvantageous effect on the person or on the patient can advantageously be effectively detected and avoided by a preferably automatic monitoring of the typical symptoms.

The stopping or the interruption of the training mode can be carried out in some exemplary embodiments automatically by the control device as a function of a signal. The signal may indicate at least one piece of information on at least one element of the group comprising a change/deviation in the muscle group distribution and in an anticyclic activity above a threshold considered to be intolerable. A disadvantageous effect on the person being trained or on the patient can advantageously be effectively detected and avoided by monitoring additional typical symptoms.

The control device may be configured in other exemplary embodiments to output parameters of the training mode and/or parameters of a breathing performance of the person and/or a piece of information on the muscle state during the duration of the training mode or subsequent to the training mode of the breathing influencing device in a form perceptible by a human being. The motivation of the patient can advantageously be increased by the outputted information and/or diagnostic information can be given to the attending physician or trainer.

Further, the control device may be configured, for example, to carry out the output optically and/or haptically and/or acoustically and related to a target variable for this person. The information to be outputted can thus be processed advantageously in an easily detectable manner for the patient, for the attending physician or for the trainer.

The control device may be configured in some exemplary embodiments to make it possible to initiate and/or to parameterize the operation of the breathing influencing device by the training person/the patient in the training mode. The support by additional persons for the preparation and the performance of the training can thus advantageously be eliminated as well as the training can be ideally adapted to the needs of the person/patient.

In some additional exemplary embodiments, the control device may be configured to automatically interrupt the training mode as a function of the information on the muscle state for a predefined time period. It is thus advantageously possible automatically to respond to temporary or milder overstraining of the muscle group being trained, without the training having to be stopped completely.

In additional exemplary embodiments, the control device may be configured to automatically determine the information on the muscle state as a function of signals that indicate an intensity of a fatigue and/or of a spasm and/or of an expiratory activity. The parameters of the person or patient, which are essential for the wellbeing, can thus advantageously also be taken into consideration in planning the training and especially when carrying out the training.

Further, the control may be configured, for example, automatically to determine an acceptable target intensity of a fatigue and/or the acceptable expiratory activity, the acceptable target intensity being configured as a target range variable over time or as a goal. A further improvement of the training can thus advantageously be achieved, because a certain intensity of the above-mentioned effect during the training is defensible and it does not cause any damage. It is advantageous, furthermore, if a training success to be expected in the course of time can already be taken into consideration during the current training.

The control device may further be configured in some exemplary embodiments automatically to determine an acceptable target intensity of a fatigue and/or of a spasm and/or of an expiratory activity, the acceptable target intensity being configured as a target range that is constant over time or as a goal. A further improvement of the training can thus advantageously be achieved because a certain constant intensity of the above-mentioned effects during the training is defensible and does not cause any damage. Thus, damage to the patient can be avoided even when the training success fails to materialize.

The control device may further be configured in exemplary embodiments automatically to allow the patient to receive feedback on a course of training and/or on the fact that a goal has been reached by maneuvers of the breathing influencing device. A person or patient, whose perception is greatly limited, can thus advantageously be informed of the result of the training and his motivation and acceptance for the training can thus be improved. This feedback is preferably carried out in an interactive or practical or acoustic form. It is preferably unnecessary for the person or the patient to detect a display visually on a display unit.

The control device may be configured in some exemplary embodiments automatically to determine the information on the muscle state as a function of signals that comprise information on the diaphragm and/or the intercostal muscles and/or antagonists, and/or additional accessory inspiratory muscles, which may be configured in exemplary embodiments as the sternocleidomastoid muscle. Overstraining of the person or of the patient can advantageously be better avoided by taking these additional parameters into consideration, and a change in the posture of the patient can be distinguished from anomalies of the inspiratory muscles.

In other exemplary embodiments, the control device may be configured automatically to determine the information on the muscle state as a function of a pneumatic signal. The breathing influencing device can thus take further essential parameters of the state of the person or patient into consideration, which enhances the reliability of the prognosis of the muscle state and makes it possible to determine additional information on the muscle state. This makes it possible, in particular, to determine the neuromuscular efficiency (Pₘᵤₛ/EMG ratio, i.e., the efficiency of how well the body converts electrical signals into activity of the inspiratory muscles), which is currently the subject of much study and research. Furthermore, this makes it possible to determine the “pressure conditions in the lungs,” which were mentioned farther above.

The control device may be configured in some other exemplary embodiments automatically to carry out an evaluation, weighting and summary when determining the information on the muscle state for at least some signals. The training mode is determined as a function of the summarized signals. Parameters especially important for the person or for the patient can thus advantageously be weighted more strongly or unimportant parameters can be weighted with a lower weight or not at all. The breathing influencing device can thus be adjusted better to the state of the person/patient.

For example, the control device may further be configured automatically to carry out a diagnostic maneuver concerning the muscle state of the person by a targeted stressing. The diagnostic maneuver may comprise in exemplary embodiments a predefined control of the breathing influencing device, which control is limited in time and differs from the normal mode. It is thus possible to use an additional mode of operation of the device, which may be used, for example, to set the training parameters for a person or which facilitates the diagnosis for the attending physician as well as can increase the precision with which the information on the muscle state is determined even more.

The control device may further be configured in some exemplary embodiments to output the results of the diagnostic maneuver and/or information on the muscle state and/or parameters of a breathing performance, optionally always related to target values for the person, in a form perceptible by a human being and/or to output parameters of the training mode. A variance comparison can thus advantageously be carried out by the training person, by the trainer, by the patient or by the attending physician, and the variance comparison can be determined in reference to a training plan.

Further, exemplary embodiments create a breathing unit for ventilating a person, comprising a device for influencing the inspiratory muscles of the person according to the present invention and/or according to one of the above exemplary embodiments, wherein the control device is further configured to operate the breathing influencing device as a function of the information on the muscle state at a first time in a normal mode with a first training intensity and at a second time in a training mode with a second training intensity. Further, the control device is configured to adapt the training mode and the second training intensity during the normal mode of the breathing influencing device as a function of the information on the muscle state for future second times. The same device can thus advantageously be used alternatively for ventilating patients and for training, which saves at least the cost of the apparatus, because many components, for example, the EMG sensors, mouthpieces, etc., can be used together. Furthermore, a supportive ventilation and an inspiratory muscle training can be carried out optionally with the same device, preferably without the patient having to be separated from the device between times.

In some other exemplary embodiments, the control device may further be configured to carry out the training mode as a change in the breathing assist of the person over a limited time for training the inspiratory muscles of the person. A training mode can thus advantageously be inserted into the phases of ventilation for restoring the inspiratory muscles, because the inspiratory muscles are atrophied already after a short time during the ventilation of the patient and breathing without the device may not possibly be sufficient any longer.

Further, exemplary embodiments create a process for influencing the inspiratory muscles of a person, comprising a detection of an electromyographic signal of the person, generation of information on the muscle state of the inspiratory muscles of the person based on the electromyographic signal, and operation of the breathing influencing device as a function of the information on the muscle state in a training mode that is limited in time. The training mode can thus advantageously be planned by taking into account the muscle state and also corrected during the performance.

Moreover, exemplary embodiments create a program with a program code for carrying out the process according to the above exemplary embodiment when the program code is executed on a computer, on a processor or on a programmable hardware component. The training mode can thus advantageously be planned and also corrected during the performance.

The program with the program code is configured as follows: When the program code is executed on a programmed or programmable device, the following steps are carried out:
An electromyographic signal from the person is detected.
At least one piece of information on a muscle state of an inspiratory muscle is generated, the information being generated on the basis of the electromyographic signal.
A breathing influencing device is controlled automatically, depending on the information generated on the muscle state and in a training mode limited in time.

Further exemplary embodiments create a program with a program code. When the program code is executed on a computer, on a processor or on a programmable hardware component, the program elicits at least the following steps: Detection of an electromyographic signal of a person; generation of information on a muscle state of an inspiratory muscle of the person based on the electromyographic signal; and control of a breathing influencing device as a function of the information on the muscle state in a training mode that is limited in time.

Further advantageous embodiments will be described in more detail below on the basis of the exemplary embodiments shown in the drawings, but the present invention is not limited generally as a whole to these exemplary embodiments. The various features of novelty which characterize the invention are pointed out with particularity in the claims annexed to and forming a part of this disclosure. For a better understanding of the invention, its operating advantages and specific objects attained by its uses, reference is made to the accompanying drawings and descriptive matter in which preferred embodiments of the invention are illustrated.

### BRIEF DESCRIPTION OF THE DRAWINGS

In the drawings:

FIG. 1 is a schematic view showing an exemplary embodiment of a simplified device for influencing the inspiratory muscles of a person;

FIG. 2 is a schematic view showing another exemplary embodiment of a device for influencing the inspiratory muscles of a patient, which device is connected to a person;

FIG. 3 is a schematic diagram for determining an acceptable intensity range for different parameters including a preference weighting;

FIG. 4 is a schematic view showing another exemplary embodiment of a simplified device for influencing the inspiratory muscles of a person with input device and output device;

FIG. 5 is a schematic view showing another exemplary embodiment of a refined device for influencing the inspiratory muscles of a person;

FIG. 6 is a refined block diagram of the interaction of the modules of the device for influencing the inspiratory muscles during a training session;

FIG. 7 is a flow diagram showing a process for influencing the inspiratory muscles;

FIG. 8 is a graph showing a work of breathing diagram;

FIG. 9A is a view showing displays of results of training sessions on a display screen;

FIG. 9B is a view showing a display of details of a training session; and

FIG. 10 is a refined block diagram of a muscle state monitoring unit.

### DESCRIPTION OF PREFERRED EMBODIMENTS

Referring to the drawings, different exemplary embodiments will be described below in more detail with reference to the attached drawings, in which some exemplary embodiments are shown.

Identical reference numbers may designate identical or comparable components in the following description of the attached figures, which show only some exemplary forms of exemplary embodiments. Further, summary reference numbers may be used for components and objects that are present multiple times in an exemplary embodiment or in a drawing, but are described together with reference to one or more features. Components or objects that are described with the same reference numbers or with summary reference numbers may have the same configuration but possibly also different configurations in respect to individual features, a plurality of features or all features, for example, their dimensioning, unless something different appears explicitly or implicitly from the description.

Even though exemplary embodiments may be modified or varied in different ways, exemplary embodiments are represented in the figures as examples and will be described herein in detail. It should, however, be clarified that exemplary embodiments are not intended to be limited to respective disclosed forms, but exemplary embodiments shall rather cover all functional and/or structural modifications, equivalents and alternatives, which are within the scope of the present invention.

It should be noted that an element that is described as being “connected” or “coupled” to another element may be connected or coupled to the other element directly or that elements located between them may be present. If, by contrast, an element is described as being “connected directly” or “coupled directly” to another element, no elements located between them are present. Other terms, which are used to describe the relationship between elements, shall be interpreted in a similar manner (for example, “between” versus “directly between,” ‘adjoining” versus “directly adjoining,” etc.).

The terminology that is being used here is used only to describe certain exemplary embodiments and shall not limit the exemplary embodiments. As being used here, the singular forms “a,” “an” and “the” shall also imply the plural forms unless the context unambiguously indicates something else, i.e., especially such that it should be read as “at least one.” Further, it should be clarified that the terms such as, for example, “contains,” “containing,” “has,” “comprises,” “comprising” and/or “having,” as being used herein, indicate the presence of said features, integers, steps, work procedures, elements and/or components, but they do not rule out the presence or the addition of one or even one or more features, integers, steps, work procedures, elements, components and/or groups thereof. The formulation A and/or B comprises only A, only B as well as A and B, unless something different is indicated.

Unless defined otherwise, all the terms used here (including technical and scientific terms) have the same meaning that a person of average skill in the art, to which the exemplary embodiments belong, attributes to them. Further, it should be clarified that terms, for example, those that are defined in commonly used dictionaries, are to be interpreted such as if they have the meaning that is consistent with their meaning used in context with the relevant technique, and they are not to be interpreted in an idealized or excessively formal sense, unless this is expressly defined here.

Exemplary embodiments for EMG-based inspiratory muscle training will be described below.

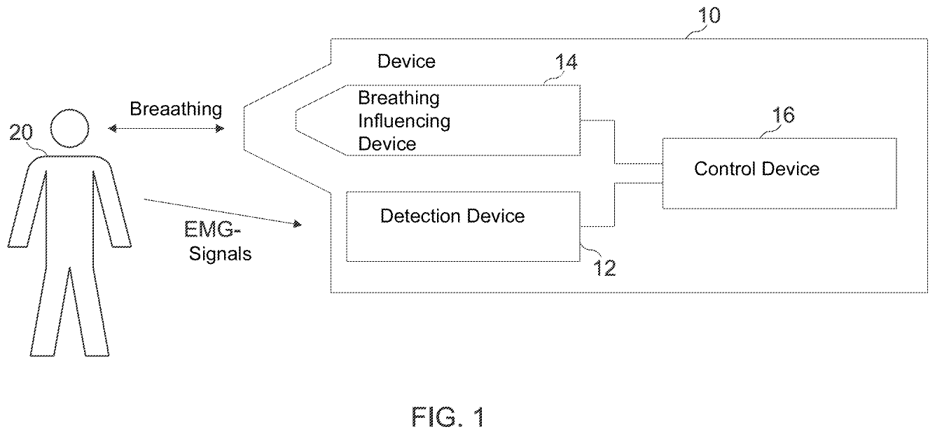

FIG. 1 illustrates an exemplary embodiment of a device 10 for influencing the inspiratory muscles of a person 20, with a detection device 12 for detecting an electromyographic signal of the person; with a breathing influencing device 14; and with a control device 16 for controlling the detection device 12 and the breathing influencing device 14. The control device 16 is configured to determine a piece of information on a muscle state of an inspiratory muscle of the person on the basis of the electromyographic signal. The control device 16 is further configured to operate the breathing influencing device 14 as a function of the information on the muscle state in a training mode, which is limited in time, with a training intensity.

The detection device 12 may comprise, for example, one or more sensors, sensor elements, electrodes or electrode pairs, e.g., needles, fine wire, electrodes, surface electrodes, pressure sensors, volume sensors, flow sensors, and gas concentration sensors, which provide corresponding sensor signals or information. The respective sensors may optionally be coupled in some exemplary embodiments with a corresponding electronic analyzing unit and provide correspondingly processed signals, for example, amplified and/or filtered and/or smoothed signals.

Further, the detection device may comprise in exemplary embodiments an amplifier with analog/digital converter. In addition, a preprocessing of the EMG signals may take place in exemplary embodiments. This preprocessing may comprise a baseline filter for offset elimination, removal of EKG artifacts and/or removal of power supply artifacts (especially 50/60 Hz) (powerline artifacts).

The device 10 further comprises the breathing influencing device 14. The breathing influencing device 14 may bring about, for example, a difficulty of breathing, which is brought about by contracted cross sections or controlled flaps or the like. The degree of difficulty is adjustable from a slight to a maximum difficulty of breathing, and in one embodiment it can be set automatically by the device 10 according to the present invention. Furthermore, the breathing influencing device 14 may comprise a plurality of sensors, which detect the extent of the breathing difficulty and convert it into signals. Moreover, the breathing influencing device 14 may also detect pneumatic parameters, e.g., the quantity and the velocity of the inhaled and exhaled breathing air of the training person or of the patient.

The breathing influencing device 14 comprises, for example, at least one of the components of an inhalation part and of an exhalation part. The inhalation part makes available to the patient 20 the gas mixture or the ambient air for inhalation. The inhalation part optionally has two gas ports (for oxygen and for compressed air), which may be connected to a local gas supply unit. As an alternative, the gas ports may also be connected to a central gas supply. An air-oxygen mixture with a defined percentage of oxygen can be provided in the downstream gas mixing unit. The gas is made available to the patient by the inhalation valve. A flow measurement device is arranged upstream of the inhalation valve. In addition, the pressure is measured by the detection device, for example, by means of a pressure sensor after the valve. The exhalation part makes it possible for the patient to exhale and releases the exhaled air, for example, into the environment. The exhalation part ensures that a minimal pressure (PEEP) is always maintained in the lungs.

As is also shown in FIG. 1, the device 10 comprises, moreover, a data-processing control device 16, which is coupled to the detection device 12 and to the breathing influencing device 14. The control device 16 receives and automatically processes signals from the detection device 12 and it automatically controls the breathing influencing device 14.

The control device 16 may correspond to any desired controller or processor or to a programmable or programmed hardware component. For example, the control device 16 may also be embodied as software, which is programmed for a suitable hardware component. The control device 16 may thus be implemented as a programmable or programmed hardware with software adapted according to the present invention. In one embodiment, the present invention may be embodied by corresponding software on an already existing hardware component. Any desired processors, especially digital signal processors (DSPs), may be used. Exemplary embodiments are not limited to a certain type of processors. Any desired processors or even a plurality of processors may be provided for embodying the control device 16.

The training mode for the inspiratory muscle training is employed in a training session. Each of the training sessions comprises an initial phase of analysis and a subsequent training phase. The training mode has the task of strengthening the inspiratory muscles, which can be achieved in many different ways.

Further, the control device 16 may also comprise in exemplary embodiments a planning unit 172 (not shown in FIG. 1). This plans the next training session automatically. Should the planning unit 30 contain information on the muscle state that contraindicates the training, the next training phase may also be automatically postponed or even be eliminated altogether. The data of the preceding training phase may also be used instead of an additional analysis phase during recurring training sessions.

The training task determined by the planning unit 172 is carried out during the training session/the training mode. A muscle state monitoring unit 174 of the control device 16, which monitoring unit is not shown in FIG. 1, has in this connection the task of permanently monitoring the state of the patient and especially of the muscles and to give recommendations for the adaptation of the training mode, training intensity and training duration.

Especially the following aspects may be taken into consideration as muscle states: Fatigue, neuromuscular efficiency, muscle regeneration quantified, for example, by DOMS (Delayed Onset Muscle Soreness), electromuscular delay, risk of atrophy, change/deviation in the muscle group distribution, an expiratory activity, an inspiratory activity, an anticyclic activity, harmful pressure conditions in the lungs, sustained spasms, cough or hiccup, muscle fatigue above a preferably predefined threshold that is considered to be intolerable, neuromechanical efficiency and maximum force as well as detected pathological states, which damage the muscle.

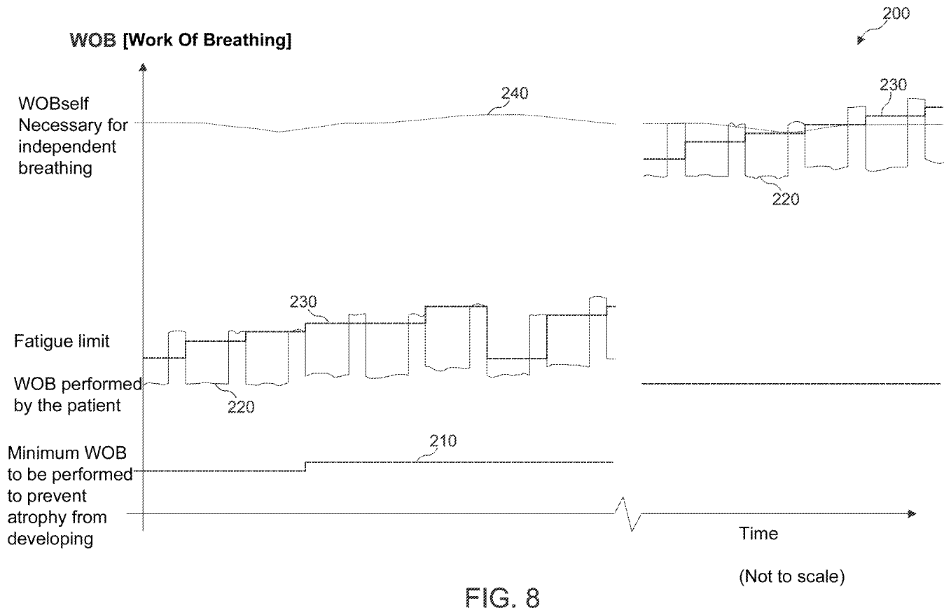

Harmful pressure conditions in the lungs cause the pulmonary pressure to move out of a predefined range, which is detected automatically. This predefined range may be configured depending on the pulmonary conditions of the patient and thus assume different values and be set automatically while the patient is connected to the device 10. Harmful pressure conditions may be based in exemplary embodiments on a fatigue limit of the patient, which may in turn be based on the highest work of breathing (WOB) of the patient or person, which the patient or the person themself can perform at the most, as it will be explained in more detail later in connection with FIG. 8.

Electromyographic signals are detected by sensor elements, which are arranged on the person or on the patient. These sensor elements transmit their data to the device 10, and the data transmission may take place by means of line connection, wireless connection, optical connection or infrared connection, etc., or it may also comprise a mixture of said transmission methods. A line connection is shown.

The electromyographic signal, or also the electromyographic lead, can be determined invasively (especially by a sensor in the trachea) or non-invasively (especially by electrodes on the skin). The EMG activity of one or more inspiratory muscles or accessory inspiratory muscles, for example, of the diaphragm, of the intercostal muscle and/or of the accessory inspiratory muscles can be recorded or detected. The detection device 12 may comprise one of the above-mentioned sensor elements, and the sensor element for the invasive or non-invasive, i.e., superficial, detection of the electromyographic signal is configured on the outer skin surface or is configured by means of the outer skin surface of the patient 20. For example, surface electrodes or electrode pairs may be arranged on the skin surface of the person or of the patient in order to detect the electromyographic signal. The signal can then be determined on the basis of measured values of at least one sensor, and the sensor used or each sensor used is arranged on the outer skin surface, i.e., on the person on the outside or at the patient, outside the body openings, such as the mouth, nose, ears, rectum, without intubation. In another exemplary embodiment, the electromyographic signal is or comprises a differential, surface-detected signal, which comprises information on a respiratory activity of at least one inspiratory muscle of the patient. The non-invasive coupling is preferred in training persons.

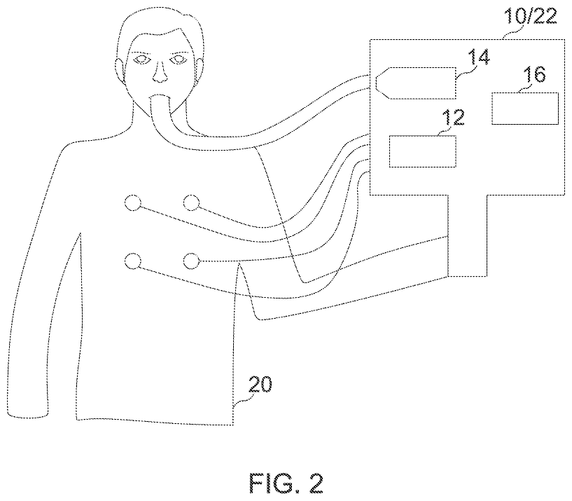

FIG. 2 shows the device 10 for influencing the inspiratory muscles in an exemplary embodiment as an inspiratory muscle training device 22 for hand-held use. The device 10 may be configured as a mobile inspiratory muscle training device 22. Furthermore, FIG. 2 shows the coupling between the person or the patient and the device 10. This coupling comprises a pneumatic coupling, by which the breath of the person or of the patient is sent through the device. This may comprise both the expiratory breathing activity and the inspiratory breathing activity. The breathing influencing device 14 is coupled to a person 20 or to a patient 20 by means of pneumatic structures, such as flexible tubes, masks, valves, branches, (endotracheal) tube, etc.

A portable device, which can be used independently from stationarily available elements, such as power, compressed air, oxygen or coupling elements to other machines and comprises in one embodiment a power supply unit of its own, can be defined as a mobile inspiratory muscle training device in exemplary embodiments. It typically has a grip or a corresponding housing design, by means of which it can be held during use and optionally also during transportation. Its weight also makes the device easily portable and its weight does not exceed 5 kg. It is equipped with input/output devices, which make it possible to operate the device and to read various parameters.

The energy supply (not shown) of the mobile inspiratory muscle training device 22 is ensured by conventional mobile energy sources, for example, by rechargeable and/or replaceable batteries. Further, the mobile inspiratory muscle training device 22 has an input/output device (not shown) for its operation and for outputting information. Moreover, a memory may be present in exemplary embodiments for storing parameters and/or earlier training results.

In other exemplary embodiments not shown in FIG. 2, the training mode is carried out as a difficulty of the breathing of the person or of the patient 20, which difficulty is limited in time and which is brought about automatically and in a targeted manner for training the inspiratory muscles. In addition to parameters of the difficulty of breathing, parameters for the duration and for the course of the training may correspondingly also be predefined in exemplary embodiments. These training courses can also be stored in the device 10, displayed and/or modified. The training mode may generally be defined as a deviation from the normal mode of the device for influencing the breathing, in which an appropriate breathing assist of the patient or of the person takes place. The device for detecting the inspiratory muscles temporarily changes the ventilation parameters during the training mode during a special monitoring of patient parameters, which may possibly indicate an overload of the patient. A detected overload of the patient or of the person may lead to a change in the training mode, which may lead to stopping, interruption and/or parameterization of the training mode.

The difficulty of breathing is achieved by pneumatic structures, and it is brought about by contracted cross sections or controlled flaps, air-permeable diaphragms or the like in breathing tubes, which the person or the patient places into his or her mouth or which are connected to corresponding mouthpieces. The degree of difficulty can be set from low to maximum difficulty of breathing. In addition, the difficulty depends on the velocity of the breath. Thus, a greater difficulty will occur at a high velocity of air of the breath than at a lower air velocity of the breath. Sensors can correspondingly determine the air velocity of the breath in the pneumatic device. The device 22 takes this into consideration when the mechanical elements used for the difficulty of breathing are set.

Breathing difficulty may also be defined as a reduction of the breathing assist, which can be made automatically available in exemplary embodiments by a ventilator. This reduction may lead in exemplary embodiments to a residual breathing assist, which does, however, already act as training for the patient. A breathing difficulty is not limited correspondingly in exemplary embodiments to a difficulty of breathing compared to free breathing without ventilator. The training may, moreover, be carried out, instead, as free breathing, i.e., as breathing without ventilator or also as reduced breathing load, which comprises now a low difficulty starting from a low breathing assist by a ventilator, the difficulty arising from the absence of the assist and from the actual breathing difficulty proper.

The training mode can be adapted adaptively in exemplary embodiments as a function of the information on the muscle state influencing of the breathing of the person or of the patient 20 by the breathing influencing device 14. As an alternative, the pressure conditions in the lungs, especially the “driving pressure,” determined from the EMG and the pneumatics, can be taken into consideration for the adaptation in exemplary embodiments. This adaptation takes place during the training, specifically by on-line analysis and/or subsequent to the training by off-line analysis.

The following steps are carried out in the course of the on-line analysis. The intensity can be optimized and adapted, optionally breath by breath. Exceptions are the detection of stopping criteria and of the performance of the resulting stopping, which may take place at any time even within one breath.

### Step 1: Recommendations Based on Individual Observations

Corresponding to the result of each of the EMG-based state signals, a recommendation is generated automatically in the first step of the on-line analysis for the further course of training, which is still independent of the other state signals in this step.

It is checked automatically within the framework of the evaluation for each state signal whether the muscles assume a state in which a further training is not meaningful—a stopping flag would be generated in such a case.

Criteria for a stopping are, for example,
A very great change/deviation in the muscle group distribution. A possible case would be, for example, when the percentage of the muscular action performed by the diaphragm drops below a low percentage in relation to the overall muscular action;
very high expiratory activity;
very high anticyclic activity;
harmful pressure conditions in the lungs, which can be detected, for example, by means of an EMG-based estimation of the pressure conditions in the respiratory system;
sustained spasms or cough or hiccup;
muscle fatigue above a threshold considered to be intolerable; and
detection of pathological states, which may damage the muscle.

In states that will foreseeably be present for a short term only, for example, sporadic spasms or cough or short-term anticyclic activity, it is also possible to recommend a short pause of a few minutes instead of the stopping flag with a pause flag before the training is restarted.

In addition to the detection of situations that suggest a training interruption or a training pause, the main task of the evaluation units is automatically to derive a training intensity to be recommended from each state signal. To make it possible to automatically find a consensus from the different state signals, not only is a concrete, preferred training intensity determined now, but an acceptable intensity range is outputted, which can be weighted corresponding to the agreement with the requirements of the respective state. Intensity ranges that are absolutely to be avoided can be permanently eliminated with a zero weighting. For example, the result of the fatigue analysis could be, for example, that the muscle is not currently exhausted, i.e., both an increase and a lowering of the training intensity are, in principle, acceptable. At the same time, the analysis of the relative activation of the diaphragm and intercostal muscles could show that the ratio of the two in relation to one another is not optimal currently, but also not critical. Consequently, a slight reduction of the intensity should be preferred; a slight increase would likewise be acceptable, but rather undesirable. The weighting can be carried out in different ways, for example, by means of representation of fuzzy sets and/or by the application of fuzzy logic or fuzzy control.

The previous training course may, in particular, also be taken into consideration when determining the intensity recommendations, in order to estimate by means of comparison with past training courses the effect that an intensity change of a defined magnitude could have on the different muscle state parameters.

### Step 2: Weighting of the Recommendations Based on the Individual Observations

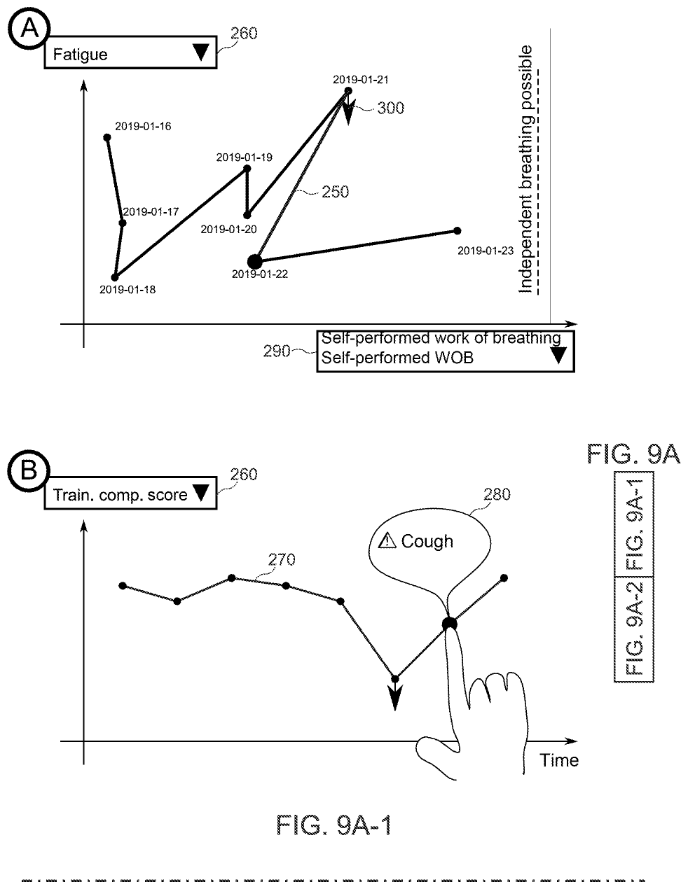

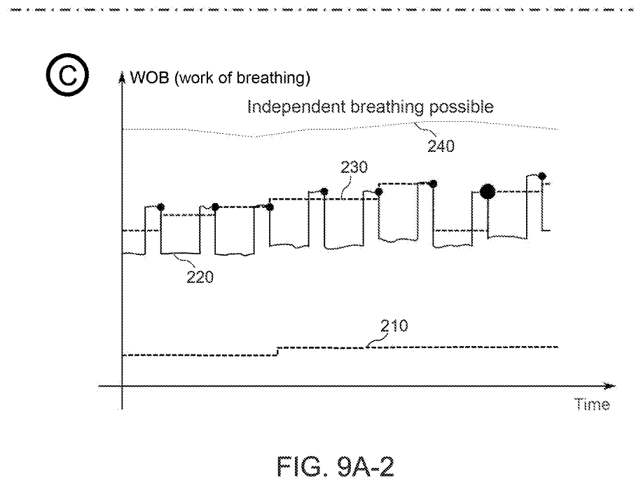

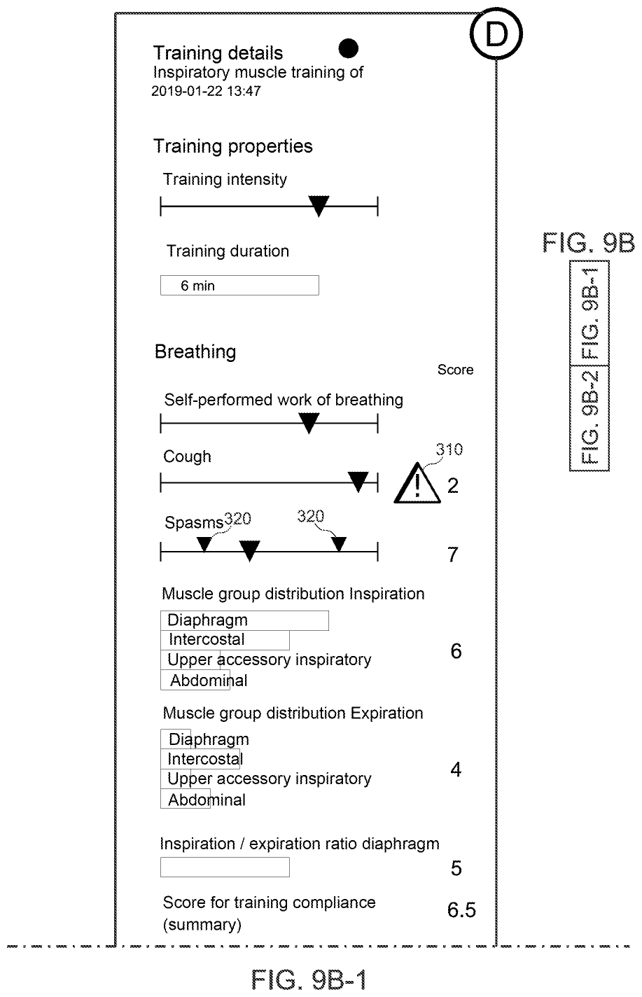

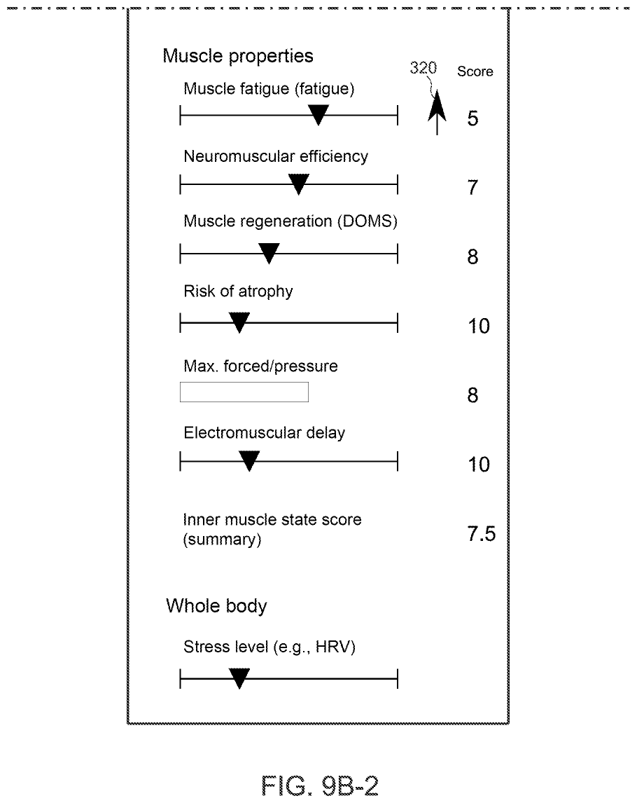

The recommendations of the different observation units are weighted automatically relative to one another in the second step of the on-line analysis in order to control the influence of the different observations on the final intensity recommendation. For example, a greater influence on the final recommendation could thus be attributed to the detection of spasm than to the detection of fatigue. Missing state information can remain unconsidered (weighting: zero) within the framework of the weighting. A user in FIG. 9B-1 (part D, training details) can influence in exemplary embodiments both the acceptable value range and the weighting over the graphic user surface and thus parameterize the evaluation of the training.

The weightings of the individual observations may depend now on all other observations; for example, it is not meaningful to take the EMG magnitudes into consideration if spasms or cough develop; the weighting of the observation of the magnitudes should therefore be reduced in this case. Furthermore, a used fatigue index may yield, for example, incorrect information when muscle re-recruitments occur and it should therefore be weighted less strongly in this case.

A possible special case of the above-mentioned general process is to set all weightings except for one weighting at zero in order to carry out the training control only on the basis of a single variable. For example, it is thus possible to carry out processes in which the training intensity is adapted (regulated) automatically such that the person/the patient must produce a fixed, predefined EMG amplitude or a fixed, predefined work of breathing.

Step 3: Combination of all Information into a Final Intensity Recommendation

A final recommendation is made in the third step for changing the training intensity on the basis of the weighted intensity recommendations, which are based on the individual observations. An optimization problem is solved for this purpose, and the training intensity shall be selected such that it has the highest possible weighting (i.e., it follows the individual recommendation as closely as possible) in the largest possible number of recommendations, under the secondary condition that it must be at least acceptable in each of the isolated recommendations. In addition, it can be taken into consideration that a) higher training intensities are to be preferred (in order to maximize the training stimulus), and b) deviations from the existing training intensity are, in principle, to be avoided, in order to make possible the most constant training sessions possible and not to confuse the person/the patient. On the other hand, it may also be desirable to prefer individual intensity changes in order to a) make a variable training possible, and b) to make it possible to investigate the effect of such changes in intensity on the muscle state.

It may be meaningful for the on-line analysis when solving the optimization problem to prevent deviations from the existing training intensity as much as possible in order to make possible training sessions that are as constant as possible.

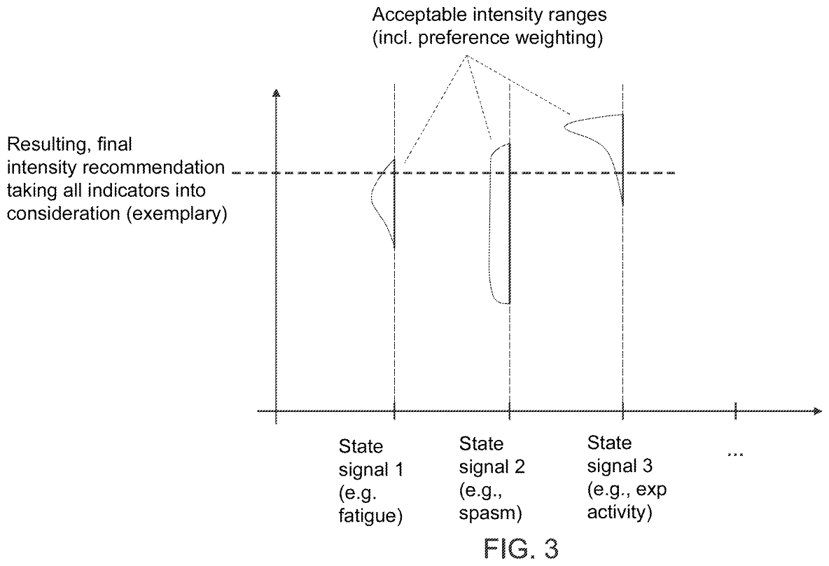

FIG. 3 visualizes the determination of an acceptable intensity range for each individual observation, including preference weighting. The state signal 1 shows as individual observations a fatigue or also muscle fatigue, which was explained in more detail at the beginning. The state signal 2 shows a spasm parameter, i.e., an indicator of the spasmodic state of the inspiratory muscles. The state signal 3 shows an indicator of the expiratory activity, i.e., an indicator of the exhalation by the person or by the patient. Each state signal has an acceptable intensity range, so that each state in the training is tolerable to a certain extent. This acceptable intensity range is shown as an area along the training intensity. The view further comprises a preference weighting of the respective state signal, so that more important states can be weighted more heavily, while unimportant states can be weighted more lightly. These weighted states are linked now in order to arrive at a resulting, final intensity recommendation, taking into consideration all indicators or states. This is shown as a dashed line to mark the resulting training intensity.

In other exemplary embodiments, not shown in FIG. 3, the control device 16 may be configured to influence the training mode concerning a training duration. The determination of the training intensity is described in detail within the framework of the on-line analysis explained above. In addition, the on-line analysis can also set the duration of the training, which is correlated especially with the training intensity. A longer training duration can correspondingly be set in case of trained muscles than in case of weak inspiratory muscles.

The control device 16 may be configured in other exemplary embodiments to stop and/or to interrupt the training mode as a function of the information on the muscle state. Simultaneously with the calculation of the intensity change recommendation for the next breath, there may be a unit that can demand a stopping of the training session just taking place. It is appropriate for this unit to be active (quasi) continuously and thus to be also able to signal a stopping during a breath. The information on stopping can be signaled from individual evaluation units and is OR-linked. The individual evaluation units can correspond to the state signals of FIG. 3 and comprise fatigue, spasm and expiration, but they are not limited to these parameters. It is sufficient here for the signal to come from one of the evaluation units. In the case of interruption, the duration of the interruption may depend on the strength of the respective state signal. As an alternative, it may also be predefined as a constant parameter.

In other exemplary embodiments, the stopping or the interruption of the training mode may be carried out as a function of a signal, which indicates information on at least one element of the group of a change/deviation in the muscle group distribution, in an expiratory activity, in an inspiratory activity, in an anticyclic activity, in harmful pressure conditions in the lungs, sustained spasms, cough or hiccup, muscle fatigue above a threshold considered to be intolerable, and detection of pathological states that can be harmful to a person. The intercostal muscles, the diaphragmatic muscle, other accessory inspiratory muscles as well as the antagonists (for example, the abdominal muscles, and the rectus abdominis muscle) may be taken into consideration here for the muscle group distribution.

The interruption may optionally be coupled with a setting of a time for the next training. The above-mentioned parameters may be taken into consideration now. A prognosis can correspondingly be made for the necessary recovery time to be expected at the end of a training based on the analyses, and the next training can be set. Moreover, it is optionally also possible to check during the normal operation based on the EMG data whether the recovery of the muscle has advanced to the extent that a further training appears to be meaningful. Taking the current state of the patient into consideration, the set training time can thus be corrected once again.

The control device 16 may be configured in exemplary embodiments to output parameters of the training mode and/or parameters of a breathing performance of the person or of the patient 20 during the duration of the training mode or subsequent to the training mode of the breathing influencing device 14. This output may also be configured as an input/output device and is used especially to give the person/the patient feedback about his performance. Moreover, the output device may be configured for a trainer or for an attending physician, which may comprise a separate input/output unit. Moreover, the input/output unit may also comprise a machine interface, for example, in the form of a plug or of a wireless interface or of a software interface.

The duration of the training mode may arise from a predefined time or it may be event-controlled. The duration of the training mode may also be given in exemplary embodiments by a combination of the predefined time and an event. For example, it is thus possible to wait for the onset of the event at most for the predefined duration. If the event fails to occur prior to the end of the predefined duration, the training mode is ended after the end of the duration. Events may occur as a function of the muscle state of the patient or of the person.

It is thus possible on the basis of the EMG-based information on the current muscle state as well as on the neuronal muscle activation produced to give the patient individual feedback in real time on the training course. The following information may be comprised: How close is he or she to reaching the goal of the training? Has the strength and/or the neuromuscular efficiency of his or her muscles increased or decreased compared to previous training sessions? This feedback may be effected, for example, visually by means of a display, acoustically, haptically or by a combination thereof. Moreover, the person or the patient may optionally receive feedback on the training course and/or on whether the goal is reached by maneuvers or special ventilation artifacts of the ventilator. Furthermore, the patient shall be able to have the ability to initiate training sessions as well as to modify the duration and the intensity of the training independently when desired via a suitable input or inspiratory muscle training device. This can take place, for example, via a usual touch display or else also via more special input devices, such as a key or a pressure ball, which can be pressed by the patient with a certain rhythm in order to initiate a training session. It would likewise be conceivable that the patient can initiate a training session based on a defined breathing rhythm, or special breathing maneuvers.

The maneuvers may be carried out as additional or missing ventilation strokes. They may also comprise usual ventilation strokes with a significantly larger or smaller quantity of air, also called ventilation amplitude. They may also comprise a temporary interruption of the ventilation as well as a change in the ventilation frequency. The maneuvers may be considered, in general, to be feedbacks for the patient, which may also be carried out in exemplary embodiments in the non-visual area. These may be haptic or tactile feedbacks for the patient or for the training person.

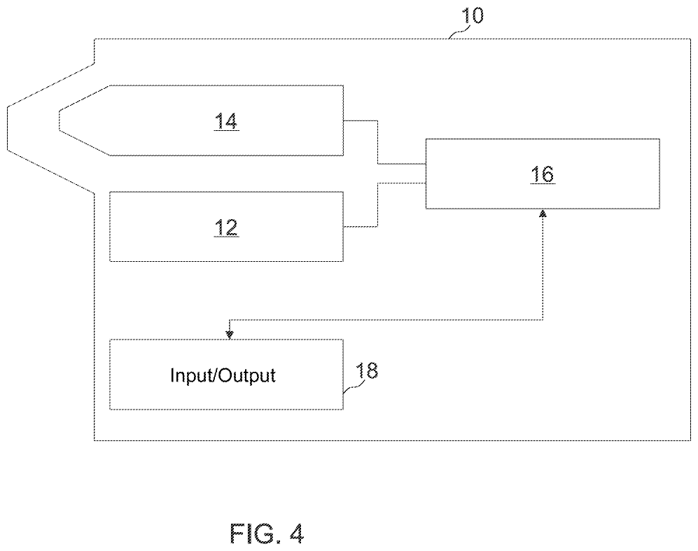

FIG. 4 shows the device 10 expanded by an input/output device. This may be configured both for a machine coupling to other devices and as one or more interfaces for the human interaction, so-called man-machine interfaces.

The control device 16 may be configured in exemplary embodiments to carry out the output 18 optically and/or haptically and/or acoustically and relative to a target variable for this person or for the patient 20. The output may comprise now the above-mentioned parameters of the training mode and/or parameters of a breathing performance of the person or of the patient 20, which are outputted during the duration of the training mode of the breathing influencing device 14. An optionally interactive display screen, signal lamps or the like may be used as an optical output. A vibration device, a change in the temperature or in the shape of a hand-held apparatus, for example, of a ball, may be used as a haptic output for patients. Acoustic outputs may be configured in the form of signals or also as speech output. Further, it is also possible to use acoustic-visual outputs or especially ventilation artifacts of the hand-held device. The output can thus be effected in a form perceptible for human beings.

The control device 16 may be configured in other exemplary embodiments to make it possible for the person or for the patient 20 to initiate and/or to parameterize the operation of the training mode of the breathing influencing device 14. This can be carried out especially by means of an operating element of the device 10 when the device 10 is configured as a training device. If the device additionally also comprises a ventilation device, a separate inspiratory muscle training device may also be configured for the patient. This inspiratory muscle training device may also comprise the above-mentioned input/output device, so that a remote control of the device is available to the patient. This remote control may be limited to a partial function of the device 10, especially to such functions that make it possible to initiate and to parameterize the training mode.

The control device (16) may be configured in additional exemplary embodiments to offer feedback on a training course and/or on reaching a goal to the patient by maneuvers of the breathing influencing device (14). These maneuvers may be configured as specific ventilation strokes, as explained in more detail in the introduction. They can correspondingly also be detected by patients, whose perception is greatly limited. Motivation of the patient and/or feedback to the patient themself can thus be achieved in such cases.

The control device 16 may be configured in other exemplary embodiments to determine an acceptable goal intensity of a fatigue and/or of a spasm and/or of an expiratory activity, the acceptable goal intensity being configured as a target range or goal that is constant over time or is variable over time. As it was explained in connection with the on-line analysis, target ranges for, for example, fatigue, spasm or expiration can be taken into consideration during the training mode. Two special cases of the on-line analysis are possible in this connection: a) The selection of an individual goal instead of a target range (pulse weight function) in order to force a defined value of an observation as a goal and b) the selection of target ranges variable in time for the different observations in order to set, for example, the target value of a steadily increasing fatigue index.

The control device 16 may be configured in additional exemplary embodiments to determine the information on the muscle state as a function of signals, which comprise information on the diaphragm and/or on the intercostal muscles and/or on antagonists. Especially the abdominal muscles, the rectus abdominis muscle, may be considered to be an antagonist in this case.

In other exemplary embodiments, the control device 16 may be configured to determine the information on the muscle state as a function of a pneumatic signal. The pneumatic signal can be detected as a breath signal of the patient which is typical of the human breathing cycle. Exemplary embodiments can thus possibly enable a less complex detection of the pneumatic signal or a detection of the pneumatic signal in the most natural manner possible. The detection device may be configured, for example, to detect the pneumatic signal as a pressure signal or as a volume flow signal at the person or at the patient. Exemplary embodiments can thus make it possible to use ventilation masks with standard components, which are configured as sensors. The detection device may be configured at least in some exemplary embodiments to detect the pneumatic signal as an expiratory and/or inspiratory breath signal of the patient. Depending on the further signal processing concept, exemplary embodiments can thus allow an analysis of the expiratory and/or inspiratory breath signals of the patient. A breath signal of a person or of a patient shall be defined here as a signal that comprises information on the breath, such as pressure, volume or concentrations, for example, oxygen O₂, carbon dioxide CO₂, water H₂O, etc.

The control device 16 may be configured in exemplary embodiments to carry out an evaluation, weighting and summary during the determination of the information on the muscle state for at least some signals, and to determine the training mode as a function of the summarized signals. The weighting may be carried out by means of a representation as fuzzy sets and/or by the application of fuzzy logic or fuzzy logic control.

In further exemplary embodiments, the control device 16 may be configured to carry out a diagnostic maneuver concerning the muscle state of the person or of the patient 20 by targeted stressing. The diagnosis can be carried out, in particular,
a) By the selection of an individual goal instead of a target range (impulse weighting function), in order to force a defined value of an observation as a goal.
b) The evaluation and isolated treatment of a subgroup or of individual groups, which take into consideration at least one member of the group comprising the expiratory activity, an inspiratory activity, an anticyclic activity, harmful pressure conditions in the lungs, sustained spasms, cough or hiccup, muscle fatigue, and pathological states. Individual muscle groups and, in an isolated form, at least one of the intercostal muscles, of the diaphragmatic muscles, of other accessory inspiratory muscles as well as of the antagonists may be comprised. A diagnostic maneuver may be defined as a process of the device 10 that supports the detection or determination of a physical or psychological illness by the physician. The diagnostic maneuver may also take place during the normal breathing in exemplary embodiments. Further, it may also be triggered in exemplary embodiments by “naturally” occurring changes in the intensity of the patient. These include intensity changes that are carried out for other therapeutic reasons other than for an inspiratory muscle training.

In other exemplary embodiments, the control device 16 may further be configured to output the results of the diagnostic maneuver and/or parameters of a breathing performance, always relative to target values for the person 20 and/or parameters of the training mode and/or, in exemplary embodiments, the information on the muscle state. The target values may be predefined statically or dynamically. Static target values may be indications of a weaning of the patient from the device configured as a ventilation device (weaning index) or the minimum performance of the inspiratory muscles to avoid an atrophy thereof, also called atrophy index. Further, fatigue, spasm or expiratory target values, etc., are possible. Dynamically predefined target values may take into consideration the change in the inspiratory muscles of the person or of the patient. For example, a fatigue target value can thus be adapted as a function of at least the current fatigue index. This also applies analogously to the atrophy target value as a function of at least the current fatigue index.

Further, exemplary embodiments create a breathing unit 100 for ventilating a person or a patient 20, comprising a device 10 for influencing the inspiratory muscles of the person or of the patient 20, wherein the control device 16 is further configured to operate the breathing influencing device 14 as a function of the information on the muscle state at a first time in a normal mode with a first training intensity and at a second time in a training mode with a second training intensity, and wherein the control device 16 is further configured to adapt the training mode and the second training intensity during the normal mode of the breathing influencing device 14 as a function of the information on the muscle state for future second times. The breathing unit 100 may be configured in exemplary embodiments as a ventilator. The ventilator may be connected to the patient both by means of a tube and in a non-invasive manner (e.g., ventilation mask).

The normal mode comprises a ventilation of a patient, which assists the breathing in the known manner permanently, so that, contrary to the training mode, the normal mode ensures the optimal breathing of the patient in the long term in a manner that is gentle for the lungs.

The breathing unit 100 comprises at least one of the components of a gas mixing unit (oxygen and compressed air), inhalation flow measurement, an inhalation valve, a pressure measurement for inhalation, an inhalation tube, an exhalation flow measurement, an exhalation valve, a pressure measurement for exhalation, an exhalation tube, an oxygen supply, a compressed air supply, an exhalation outlet and a microcontroller unit 16, which may be coupled to the pressure and flow sensors as well as to the valves and to the gas mixing unit.

The adaptation of the training mode during the normal mode is also called off-line analysis. Contrary to the on-line analysis, the off-line analysis is always carried out only at the end of a training session. The signal curves of the individual muscle state indicators must therefore be stored intermediately within the framework of the off-line analysis. The procedure is basically similar to the on-line analysis, cf. the description of the on-line analysis above. However, there are at times different evaluation criteria, different weightings and different secondary conditions in the optimization task.

Contrary to the on-line analysis, more attention is paid in the evaluation to the course of the EMG states during the training session. What is important in this connection is to determine whether problematic states have developed over the duration of the training. Especially in case of past changes in the training intensity from one training session to the next, it can be evaluated here whether and how the change of the training task has affected the muscle state. It is investigated in this connection how the relative change in the mechanical training task behaves in relation to the relative change of the EMG performance. Further, it can be investigated whether an enhanced training task also leads to more muscle contractions (in the correct muscles). Contrary to the on-line analysis, it may be meaningful to challenge deviations from the existing training intensity to a certain extent. The training becomes more varied thereby and more information can be collected within the framework of the analysis.

In addition, the data obtained in the current normal mode may also be included in the off-line analysis. Thus, changes of the patient, for example, long-lasting throat irritation or the like, may lead to a reduction of the planned training task. On the other hand, a markedly improved EMG may, for example, enhance the planned training task.

Finally, a fundamental difference from the on-line analysis is that not only a different training intensity, but also a different training mode can be recommended in the off-line analysis. This may be useful, for example, when a patient or a person does not tolerate a defined mode well or in order to increase the variability of the training. The recommendations of the off-line analysis unit may be processed now both in an automated manner (in the planning unit) and also displayed to the user of the device (clinical staff, trainer). The decision is made in the latter case by the user of the device, supported by the recommendations provided by the analysis unit and by information.

### Update of the Individual Evaluation Criteria

In addition to the recommendation of a new training intensity, an update of the evaluation criteria is also carried out within the framework of the off-line analysis. For example, the threshold values starting from which a defined level of fatigue or a defined ratio of the diaphragmatic activity to the intercostal activity is rated as being critical can thus be adapted.

Possible reasons for the adaptation of these threshold values are a modified training intensity, a modified training mode as well as changes in the measuring methods, when, for example, the EMG electrodes are located in different positions or the conductivity of the skin has changed (for example, because of sweat). The question of the general comparability of measurements in different training sections shall also be taken into consideration in the formulation of the respective intensity recommendations.

The control device 16 may further be configured in other exemplary embodiments to carry out the training mode as a change, which is limited in time, in a breathing assist of the person or of the patient 20 for training the inspiratory muscles of the person or of the patient 20.

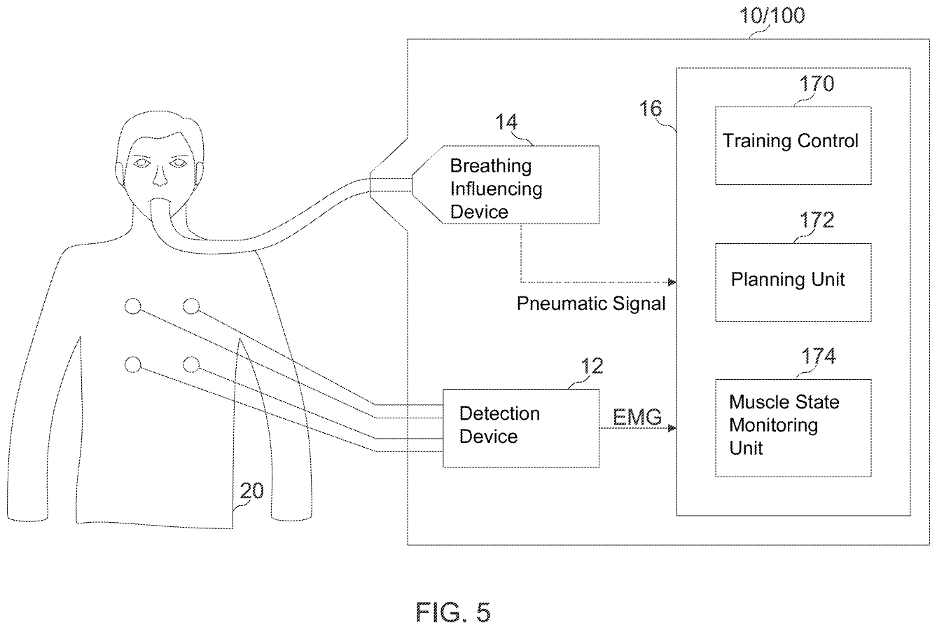

FIG. 5 describes another exemplary embodiment of a refined view of the device 10 or of the breathing unit 100 for influencing the inspiratory muscles of a person or of a patient. The device 10/100 comprises here the breathing influencing device 14, which can be pneumatically connected to the patient or to the person, which is brought about typically by means of tubes, mouthpieces, etc. The device 10/100 further comprises the detection device 12 for detecting the EMG signals of the patient or of the person. The detection device 12 sends the EMG signals to the control device 16, which optionally also receives from the breathing influencing device 14 a signal, which is based on pneumatic properties of the breathing of the patient or of the person 20. The control device 16 transmits a control signal to the breathing influencing device 14 for setting the influencing of the breathing of the patient or of the person 20. The control signal is based on analyses of the EMG signals and of the optional pneumatic signal. A training control 170 of the control device 16 generates the actuating variables and set points for the breathing influencing device 14 for the training mode. Further, a planning unit 172 of the control device 16 determines the training task for the next training mode. Finally, the muscle state monitoring unit 174 of the control device permanently monitors the state of the patient or of the person and monitors especially the relevant muscles. It gives recommendations for the adaptation of the training mode, training intensity and training duration.

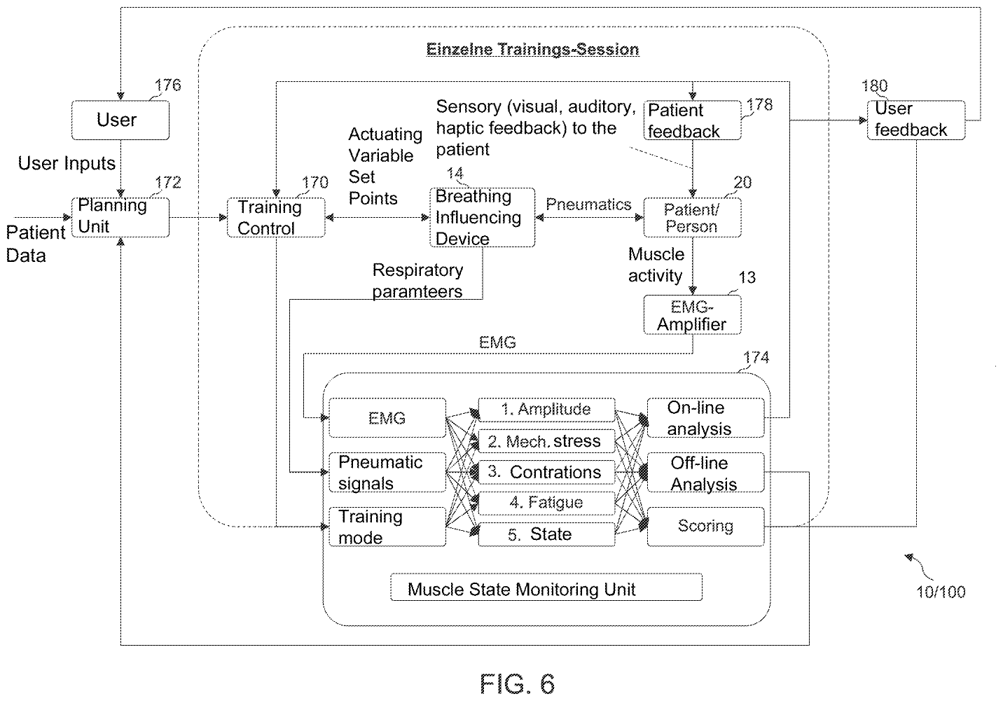

FIG. 6 describes the components of the breathing unit for the training mode, which is applied during a training session. Patient data are sent here to the planning unit 172. This additionally receives user inputs from the user component 176 and sends signals to the training control 170, which also receives signals from the on-line analysis of the muscle state monitoring unit 174. In return, the training control 170 sends actuating variables and set points to the breathing influencing device 14 as well as to the training mode of the muscle state monitoring unit 174. The breathing influencing device 14 is coupled pneumatically to the patient or to the person 20 and sends respiratory parameters for the pneumatic signals of the muscle state monitoring unit 174. The patient 20 receives feedback, also called response, from the patient feedback 178 and sends its muscle activities to the EMG amplifier 13, which sends, in turn, EMG to the EMG unit of the muscle state monitoring unit 174. The components of the muscle state monitoring unit 174 that were mentioned hitherto are linked within the muscle state monitoring unit 174 with all other components, namely, 1. amplitude, 2. mechanical stressing, 3. unusual condition, 4. fatigue and 5. state, which are linked, in turn, with all additional components, which comprise the on-line analysis, the off-line analysis and the scoring. The on-line analysis additionally sends its information to the planning unit 172. The scoring sends signals to the user feedback 180. The user feedback 180 sends information to the user component 176. The above-mentioned components may be configured as distributed or combined hardware, as software or as preprogrammed hardware elements, which contain a processor.

It shall also be stated in connection with FIG. 6 that the inspiratory muscle training consists of recurring training sessions, in which the training mode is applied. Each of the training sessions comprises an initial analysis phase and the subsequent training phase. A training task is determined for the next training phase by the planning unit during the planning phase. The training task includes:
the training modality, for example, negative minimum inhalation pressure, controlled by WOB (work of breathing), by the EMG threshold value, etc., also called training mode here,
the training intensity, which is expressed differently in the different training modalities, as well as
the duration of the training.

Should the planning unit 172 receive information on the muscle state that contraindicates the training, the next training phase may also be postponed or eliminated altogether. The previous training phases may also be used instead of an additional analysis phase in case of recurring training sessions.

The training task determined by the planning unit 172 is carried out during the training session. The muscle state monitoring unit 174 has now the task of permanently monitoring the state of the patient and especially of the muscles and of giving recommendations for the adaptation of the training modality, training intensity and training duration.

The off-line analysis unit becomes active after the training phase. It analyzes the training phase retroactively. The off-line analysis unit has the following tasks:
analysis of the past training phase in order to possibly recommend a change for the next training phase (duration, intensity, further training paths);
generation of trends and displayable data for the clinical staff; and
calculation of a ready-to-wean indicator.

The ventilation mode, which is also called normal mode, may, of course, also be adapted outside the training sessions on the basis of the results of the analysis units.

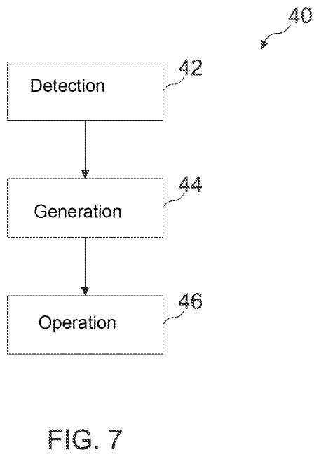

FIG. 7 shows a process for influencing the inspiratory muscles. One or more electromyographic signals of a person or of a patient are detected here in the detection block 42. Information is sent from the detection block 42 to the generation block 44, which generates information on a muscle state of an inspiratory muscle of the person 20 on the basis of the electromyographic signal. In addition or as an alternative, additional measured signals, such as pressure, volume flow (flow), volume and/or etCO₂ can be used in exemplary embodiments. Information is sent from the generation block 44 to the operation block 46, which carries out an operation 46 of a breathing influencing device 14 as a function of the information on the muscle state in a training mode, which is limited in time.

Further, exemplary embodiments create a program with a program code for carrying out the process 40 when the program code is executed on a computer, on a processor or on a programmable hardware component.

Moreover, exemplary embodiments create a program with a program code when the program code is executed on a computer, on a processor or on a programmable hardware component. The program comprises the following steps: Detection (42) of an electromyographic signal of a person; generation (44) of information on a muscle state of an inspiratory muscle of the person (20) based on the electromyographic signal; and control (46) of a breathing influencing device (14) as a function of the information on the muscle state in a training mode, which is limited in time.

FIG. 8 shows a diagram 200 with the breathing performance of the patient and with different limit values over time. The lowermost curve 210 describes the work of breathing (WOB) to be performed as a minimum by the patient in order for atrophy not to occur. Further, the WOB 220 performed by the patient as well as the corresponding fatigue limit 230 are shown. Finally, a line for WOBself 240 is shown for the necessary work of breathing for the independent breathing of the patient without ventilator.

It should also be stated in connection with the visualization of the progression of training in a WOB diagram with information on fatigue and atrophy that additional information is obtained by the EMG-assisted monitoring and analysis of the training of the inspiratory muscles. This information also offers the clinical staff extra value for estimating the state of the patient. It is, however, necessary to process and to display the data correspondingly in order to make it possible to interpret the data in a simple manner.

One possibility for this is to expand a WOBself (ready to wean) diagram over time. This is based on a graphic diagram 200, in which the work of breathing performed by the patient is shown over time. Distinction is also made now, in particular, between work of breathing performed by the patient and work of breathing performed by the breathing influencing device 14. A WOB diagram, which is enriched with additional information, will be described below. This information includes especially information from the inspiratory muscle training, information on the muscle state as well as information derived therefrom.

The individual training sessions can be seen in the diagram as temporary deflections of WOB 220. The diagram is complemented by the WOB to be expected when the patient would be breathing fully independently (WOBself) 240 as well as by a fatigue limit 230 (estimation of a maximum muscle performance in respect to imminent fatigue) and by an atrophy limit (estimation of a minimal muscle performance in respect to an imminent muscle atrophy).

The WOB necessary for the independently breathing patient (WOBself) 240 is calculated as the sum of the independently performed WOB 220 and the component originating from the ventilator. Ventilation-related extra components (for example, additional tube resistance) must have been removed before to this end from the component performed by the ventilator.

The fatigue limit 230 shown characterizes the highest WOB performed by the patient, without harmful muscle fatigue developing. The fatigue limit is calculated, as a rule, on the basis of the EMG by the analysis unit at the end of the training sessions. If fatigue (determined by an EMG-based fatigue index), which exceeds an extent that is desired and is acceptable within the framework of the training, develops during the training sessions, whereas fatigue did not develop prior to the training, the fatigue limit is set at a value between the WOB found prior to the training and the WOB found during the training. The exact level depends on the extent of the change in the fatigue index and the duration of the time elapsing until the onset. If there is no substantial change in the fatigue index during the training, the fatigue limit 230 is estimated to be slightly above the training WOB 220. Should fatigue develop even outside the training, the fatigue limit is lowered. The fatigue limit represents the maximum breathing effort that can be exerted permanently. The distance of the fatigue limit to the WOB necessary for the independent breathing (WOBself) shall be defined as the Ready to Wean Indicator. If the fatigue limit intersects WOBself and remains permanently above it, it can be assumed that the patient can breath spontaneously at least as far as the inspiratory muscles are concerned and weaning can be taken into consideration (ready to wean).

The atrophy limit 210 shown characterizes the minimum WOB component to be performed by the patient in order to prevent a muscle atrophy. If the patient WOB component is below this limit, it can be assumed that the inspiratory muscles are subject to atrophy and will continue to degenerate. Even if it is possible to infer an atrophy from the EMG, this limit is preferably derived statically from the body weight (and optionally from the age and sex, illness, temperature and other known parameters of the patient). In an alternative form, the intrinsic work of breathing performed in the medium-term past is used additionally in the calculation of the atrophy limit. The atrophy limit is then to be interpreted such that a further degeneration of the muscles can be expected based on atrophy if the atrophy is below the limit.

In an alternative diagram (not shown), the vertical axis is shown relative to the WOB necessary for the independent breathing.

It should also be stated in connection with the determination of an EMG-based ready-to-wean score that the ready-to-wean score expresses the extent to which the patient is able to breath spontaneously in a sustained manner. Contrary to a weaning indicator, which is based only on the component of the WOB that is performed by the patient themself or on other pneumatic data, the weaning indicator being presented here can incorporate the additional EMG-based information on the muscle state. As a result, it expresses not only whether the patient could perform his intrinsic work of breathing after a sufficiently short time during the training phases, but it also takes into consideration the behavior of the inspiratory muscles and thus it has a much better possibility of making a long-term prognosis of whether the patient is capable of performing the necessary work of breathing permanently.

The improved ready-to-wean score is also based, in principle, on the component of the self-performed WOB in relation to a WOB that would be necessary for a fully independent work. In addition, the score does, however, also take into account the state of the muscles during the short-term training sessions. The time at which signs showing that the load that is to be performed during the short training time periods cannot be performed permanently develop is taken into account, in particular.

Specifically, the following factors are included:
endurance/duration (the longer the patient has endured, the better),
devaluation, if fatigue developed in the process (based on EMG),
devaluation, if expiratory activity develops (e.g., based on EMG),
devaluation, if intense re-recruitment occurs (e.g., based on EMG),
devaluation, in case of spasms (based on EMG),
taking the performance distribution within one breath into consideration (based on EMG).

One would thus work continuously towards a weaning trial. A person who is ready to wean would thus be expected not to develop fatigue phenomena over the medium term even at 100% intrinsic WOB component.

FIGS. 9A and 9B show in exemplary embodiments displays on a display screen in the form of screenshots, which consist, as an example, of four parts:
Part A: A locus curve diagram of the fatigue index 250 over time, here summarized according to days. In addition, additional parameters 290 can be selected and faded in, for example, the self-performed work of breathing, the self-performed WOB;
Part B: Diagram of a value over time 270, which is shown here as an example as the training result 260 (training compliance score);
Part C: Work of breathing-based diagram of self-performed work of breathing, fatigue and atrophy, which is described in detail as FIG. 8; and
Part D: Training details for a selected training session.

The main tasks of this coherent diagram are especially a
visual communication on the results of a training session or of a diagnostic maneuver and of the state of the muscles and of the respiratory system;
visual communication of events that have led to an interruption of training;
visual communication of events that have decisively led to a change in the training intensity;
transparent presentation of the development of the evaluation of the muscle state. The user shall, in particular, be able to reconstruct how a certain result (score) was poor and what consequences have arisen therefrom.
feedback from the user for the acceptable value ranges. The user can limit/adapt the acceptable range 320. This is shown as an example in the case of “spasms” in FIG. 9B-1. It can, however, also be able to be set in exemplary embodiment in case of at least one additional value, in case of a plurality of values or in case of all values.
influence of the user on the weighting (not shown in the diagram) may also be possible via the graphic user surface.

The information shown as an example is processed, in general, such that the following aspects are taken into consideration:
The individual training sessions may be characterized such that a coupling can be established between the different parts A-D indicated. This coupling can be solved as an example by the display of rainbow-colored dots. As an alternative, shapes, numbers, blinking patterns or the like would be conceivable as marks.
The user can select interactively in exemplary embodiments which values shall be shown on the axes (suggested by dropdown menu). These may be both individual values (e.g., fatigue, EMG performance of a muscle group or WOB) derived from the EMG/pneumatic data and values/scores derived or summarized therefrom.

Moreover, all the parameters mentioned farther above can be incorporated in exemplary embodiments of FIG. 9B. This can comprise at least individual parameters, all from the following groups: 1. Amplitude, 2. mechanical stressing, 3. unusual contractions, 4. fatigue, 5. “inner” states as well as 6. additional information that is also generated with the EMG signal.

Related exemplary embodiments may lead, as an example, to the following diagrams:
an evaluated training result (score of training compliance) as a summary of the training performance, which can be derived, among other things, from the EMG performances of the muscle groups;
an evaluated training result within evaluated muscle state results as a summary of the internal information on the muscle state, which is derived from the EMG. This internal state information may comprise fatigue, neuromuscular efficiency, muscle regeneration—for example, quantified by DOMS (delayed onset muscle soreness), electromuscular delay, risk of atrophy as well as maximum pressure/force.

Additional information may be displayed in the diagrams in exemplary embodiments at the points belonging to individual training sections. This may be configured, for example, in a bubble 280 or as a local addition to the data point, which is shown here as an example as an arrow 300 pointing downward to visualize an adaptation of the reduced training intensity for the next training.

In addition, the dots of the training sessions can be connected in exemplary embodiments in the locus curve diagram 250 by fading lines, different line thicknesses, color, broken lines or the like in order to represent the time information. Furthermore, the dots may be additionally characterized by additional information in the locus curve diagram 250 and thus they can be complemented, for example, with the calendar date at which they were detected.

The display of training details as they are shown, for example, in FIG. 9B, part D, will be shown below. The training details shall serve, in principle, the following purposes:
communicating to the user further details of the training, i.e., for example, also the values that were not selected just now for the display in one of the graphs;
in addition, part D also has, however, the important task of presenting to the user the decisions of the automatic training adaptation. The user shall thus be able to reconstruct the automatic decisions. This may comprise, for example, the following:
communication of problems that have led to a stopping of the training session, which is shown, for example, as a warning triangle 310 at “cough” in the example; and
communication of effects that have decisively led to a change in the training intensity. In the example, it is the green arrow 300 at Fatigue, which is used as information that it was decided on the basis of an only very mild fatigue to increase the training intensity.

Further, training details may serve the following purposes:
the development of the weighted results (scores) can be communicated to the user in case of summarizing weighted results (scores);
individual values can be grouped.

For example, the following groups can thus be carried out:
training properties;
breathing (based mainly on EMG performance);
inner muscle properties;
properties pertaining to the entire body, which, measured, for example, as stress, may be detected by the heart rate variability.

Specifically, FIG. 9B shows as display D “training details,” which are designated as “inspiratory muscle training” with date and time. It is followed by the heading “Training properties,” which displays a training intensity graphically and indicates the training duration with 6 minutes. This is followed by the heading “Breathing.” The work of breathing performed by the patient themself is represented graphically at the very top. This is followed by individual results, which are always evaluated with a number, the score. The individual results comprise the graphically represented cough intensity “cough” with the warning triangle 310 and score 2, as well as correspondingly represented spasm intensity “spasms” with score 7. The designation and graphic representation of the diaphragmatic intensity, of the intercostal muscle, of the upper inspiratory muscles and of the abdominal muscles, which together yield a score of 6, follow under the subheading “Muscle group distribution inspiration.” This is further followed by the “Muscle group distribution expiration” with the same muscle groups and with the same graphic representation with the summary score 4. The “Inspiration/Expiration ratio diaphragm” in the graphically processed form with the score 5 concludes the breathing group. The “Score training compliance (summary)” has the value of 6.5.

Then follows the group “Muscle properties” with the designation and graphic representation of muscle fatigue (fatigue) with arrow 300 and score 5, neuromuscular efficiency with score 7, muscle regeneration (DOMS) with score 8, risk of atrophy score 10, and electromuscular delay with score 10. This list is summarized as “inner muscle state score (summary)” with score 7.5. A “Stress level (e.g., HRV)” (heart rate variability) is finally shown graphically under the heading “whole body.”

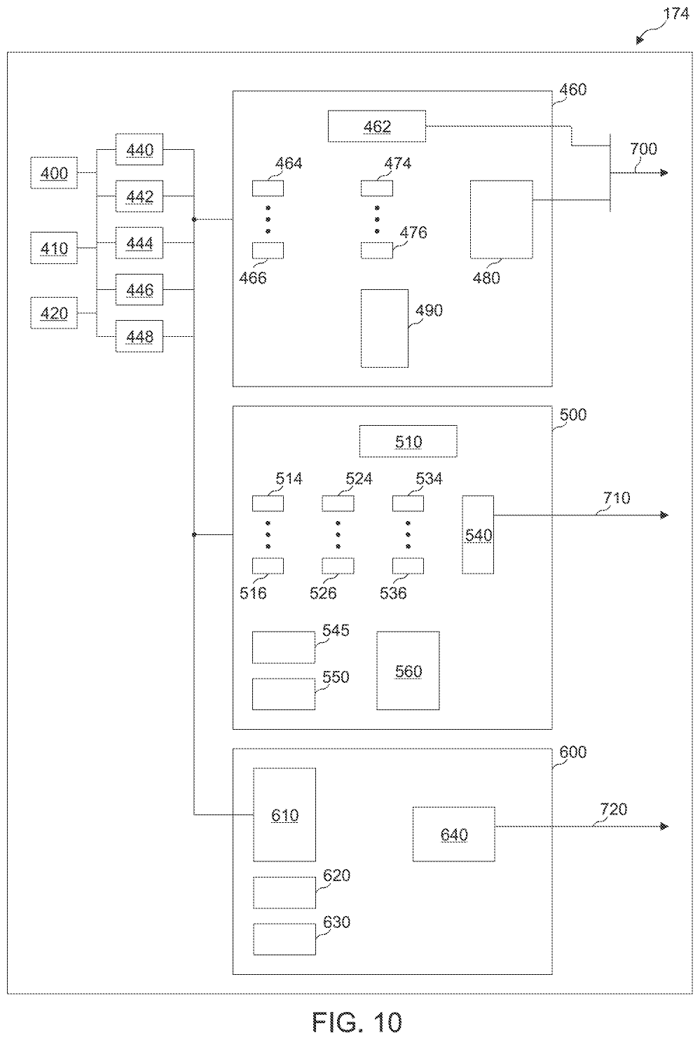

FIG. 10 shows a refined block diagram of the muscle state monitoring unit 174. EMG 400, pneumatic signals 410 and training settings 420 provide input signals here. These signals are connected to the amplitude block 440, which comprises the aspects (not shown) muscle group distribution, expiratory activity, anticyclic activity/time curve of breathing stroke as well as thoracic strain without breathing. Further, the signals are connected to the mechanical stressing block 442, which comprises the aspects breathing performance, pressure difference in the lungs, as well as counteracting forces (not shown). Further, the signals are connected to the unusual contractions block 444, which comprises the aspects spasms, cough and hiccup (not shown). Further, the signals are connected to the fatigue block 446. Finally, the signals are connected to the state block 448, which comprises the aspects pathology, atrophy, neuromuscular efficiency, muscle regeneration (quantified, e.g., by DOMS, delayed onset muscle soreness), and electromuscular delay (not shown). The output signals of the blocks 440, 442, 444, 446 and 448 are connected, in turn, to the off-line analysis block 460, to the on-line analysis block 500 as well as to the scoring unit 600.

In the off-line analysis block 460, each of the above-mentioned aspects of all blocks 440, 442, 444, 446 and 448 is connected to corresponding online evaluations 474 through 476, which are illustrated each only as two blocks for clarity's sake. Further, the on-line analysis comprises a function recommendation for training stopping/recommendation for “short” recovery phase 462, which are likewise connected to the on-line evaluations 464 through 466. A weighting determination with the taking into account of cross-relationships 490 is connected to the on-line evaluations 464 through 466 and to the on-line weightings 474 through 476 on the input side as well as to the control thereof. A summary for adaption of the training intensity during the running training unit 480 is connected to the on-line weightings 474 through 476. The latter yields on the output side together with the function 462 the signal for the training control 700.

The off-line analysis 500 comprises the functions intermediate memory for each aspect 514 through 516, which is connected to the respective aspect. An off-line evaluation for each aspect 524 through 526 is connected to the corresponding intermediate memory for each aspect 514-516. Off-line weightings for each aspect 534 through 536 are connected to the off-line evaluation for each aspect 524 through 526. A weighting determination with the taking into account of cross-relationships 510 is connected in a controlling manner to the intermediate memory for each aspect 514 through 516, to the off-line evaluation for each aspect 524 through 526 and to the off-line weightings for each aspect 534 through 536. An EMG comparability detection 545 is connected to the EMG 400. A training intensity/mode change 550 is connected to the training settings 420. An update evaluation criterion 560 is connected to the EMG comparability 545, to the training intensity/mode change 550 as well as to the off-line evaluation for each aspect 524 through 526. A summary for the adaption of the training intensity recommendation for the planning unit to the next training unit 540 is connected to the off-line weightings for each aspect 534 through 536, which delivers on the output side the signal to the planning unit/or as a recommendation to the display 710.

The scoring unit 600 comprises the functions state criteria 610, which is connected to the respective aspects. A WOB intrinsic component estimation 620 is connected to the pneumatic signals 410. A total WOB estimation (device and patient) 630 is connected to the EMG 400. A ready-to-wean score 640 is connected toe the above-mentioned three blocks 610, 620 and 630 and it provides the signal for the display 720 on the output side.

In addition, it is also possible to carry out an estimation (not shown) of the stress level of the entire body, which can be determined on the basis of the heartbeat present in the respiratory EMG by means of the heart rate variability.

The above-described exemplary embodiments represent only an illustration of the principles of the present invention. It is obvious that modifications and variations of the arrangements and details being described here will be obvious to other persons skilled in the art. The present invention is therefore intended to be limited only by the scope of protection of the following patent claims rather than by the specific details, which were presented here on the basis of the description and the explanation of the exemplary embodiments.

While specific embodiments of the invention have been shown and described in detail to illustrate the application of the principles of the invention, it will be understood that the invention may be embodied otherwise without departing from such principles.

### LIST OF REFERENCE NUMBERS

10 Device for influencing inspiratory muscles
12 Detection device
13 EMG amplifier
14 Breathing influencing device
16 Control device
18 Input/output unit
20 Person/patient
22 Device for influencing inspiratory muscles, optionally configured as a mobile inspiratory muscle training device
40 Process for influencing inspiratory muscles
42 Detection of the EMG and optionally of the pneumatic data
44 Generation of the information on the muscle state
46 Operation of the breathing influencing device
100 Breathing unit for ventilating a person
170 Training control
172 Planning unit
174 Muscle state monitoring unit
176 User
178 Patient feedback
180 User feedback
200 Energy per time diagram
210 Minimum WOB for avoiding atrophy
220 WOB performed by the patient
230 Fatigue limit
240 WOBself, for independent breathing
250 Locus curve, day-related fatigue values
260 Display of the designation of the values shown
270 Locus curve, day-related training success experience (training compliance score)
280 Detail information
290 Self-performed work of breathing, self-performed WOB
300 Adaptation of the training intensity
310 Warning triangle because of considerable coughing
320 Acceptable range, optionally can be set by the user
400 EMG
410 Pneumatic signals
420 Training settings
440 Amplitude
442 Mechanical stresses
444 Unusual contractions
446 Fatigue
448 State
460 Off-line analysis
462 Recommendation for training stopping/recommendation for “short” recovery pause
464-466 On-line evaluations for each aspect
474-476 On-line weightings for each aspect
480 Summary for adaption of the training intensity during the running training unit
500 Off-line analysis
510 Weighting determination taking cross-relationships into consideration
514-516 Intermediate memory for each aspect
524-526 Off-line evaluation for each aspect
534-536 Off-line weighting for each aspect
540 Summary for the adaption of the training intensity recommendation for the planning unit to the next training unit
545 Determination of EMG comparability
550 Change in training intensity/mode
560 Update of evaluation criteria
600 Scoring unit
610 State criteria
620 Estimation of intrinsic WOB component
630 Estimation of total WOB (device and patient)
640 Ready-to-wean score
700 Signal to training control
710 Signal to planning unit/or as a recommendation to display
720 Signal to display

1. Device for influencing the inspiratory muscles of a person, the device comprising:
a detection device for detecting an electromyographic signal of the person;
a breathing influencing device, and
a control device for controlling the detection device and the breathing influencing device,
wherein the control device is configured to determine information on a muscle state of an inspiratory muscle of the person based on the electromyographic signal, and
wherein the control device is further configured to operate the breathing influencing device as a function of the information on the muscle state in a training mode, which is limited in time, with a training intensity.

2. Device for influencing the inspiratory muscles of a person in accordance with claim 1, wherein the control device is configured to carry out the training mode as an at least temporary difficulty of breathing of the person for training the inspiratory muscles.

3. Device for influencing the inspiratory muscles of a person in accordance with claim 1, wherein the control device is configured adaptively to adapt the influencing of the breathing of the person by the breathing influencing device in the training mode as a function of the information on the muscle state.

4. Device for influencing the inspiratory muscles of a person in accordance with claim 1, wherein the control device is configured to influence the training mode in respect to a training duration.

5. Device for influencing the inspiratory muscles of a person in accordance with claim 1, wherein the control device is configured to stop or to interrupt the training mode as a function of the information on the muscle state.

6. Device for influencing the inspiratory muscles of a person in accordance with claim 5,
wherein the stopping or the interruption of the training mode is carried out as a function of a signal,
wherein the signal indicates at least one element of the group of
an expiratory activity,
an inspiratory activity,
harmful pressure conditions in the lungs,
sustained spasms,
cough or hiccup,
muscle fatigue above a threshold considered to be intolerable, and
detection of pathological states, which may be harmful to the person.

7. Device for influencing the inspiratory muscles of a person in accordance with claim 5,
wherein the stopping or the interruption of the training mode is carried out as a function of a signal,
wherein the signal indicates at least one piece of information on at least one element of the group of
a change/deviation in the muscle group distribution and
an anticyclic activity above a threshold considered to be intolerable.

8. Device for influencing the inspiratory muscles of a person in accordance with claim 1,
wherein the control device is configured to output parameters of the training mode and/or parameters of a breathing performance of the person during the duration of the training mode or subsequent to the training mode of the breathing influencing device,
wherein the control device is further configured to carry out the output optically and/or haptically and/or acoustically and relative to a target variable for this person.

9. Device for influencing the inspiratory muscles of a person in accordance with claim 8, wherein the control device is configured to initiate and/or to parameterize the operation of the training mode of the breathing influencing device by the person.

10. Device for influencing the inspiratory muscles of a person in accordance with claim 1, wherein the control device is configured to provide feedback on a training course and/or attainment of a goal by maneuvers of the breathing influencing device.

11. Device for influencing the inspiratory muscles of a person in accordance with claim 1, wherein the control device is configured to determine the information on the muscle state as a function of signals that indicate an intensity of fatigue and/or of a spasm and/or of an expiratory activity.

12. Device for influencing the inspiratory muscles of a person in accordance with claim 11,
wherein the control device is configured to determine an acceptable target intensity of fatigue and/or of a spasm and/or of an expiratory activity, and
wherein the acceptable target intensity is configured as a target range or goal that is variable over time.

13. Device for influencing the inspiratory muscles of a person in accordance with claim 11,
wherein the control device is configured to determine an acceptable target intensity of a fatigue and/or of a spasm and/or of an expiratory activity,
wherein the acceptable target intensity is configured as a target range or goal that is constant over time.

14. Device for influencing the inspiratory muscles of a person in accordance with claim 1, wherein the control device is configured to determine the information on the muscle state as a function of signals that comprise information on diaphragm muscles and/or on intercostal muscles and/or on antagonists muscles.

15. Device for influencing the inspiratory muscles of a person in accordance with claim 1, wherein the control device is configured to determine the information on the muscle state as a function of a pneumatic signal.

16. Device for influencing the inspiratory muscles of a person in accordance with claim 1, wherein the control device is configured
to carry out an evaluation, weighting and summary during the determination of the information on the muscle state for at least some signals; and
to determine the training mode as a function of the summarized signal.

17. Device for influencing the inspiratory muscles of a person in accordance with claim 1, wherein the control device is further configured to carry out a maneuver to determine the muscle state of the person by targeted stressing.

18. Device for influencing the inspiratory muscles of a person in accordance with claim 17, wherein the control device is further configured to output the results of the maneuver and/or parameters of a breathing performance always relative to target values for the person and/or parameters of the training mode.

19. Breathing unit for ventilating a person, comprising a device for influencing the inspiratory muscles of the person, the device comprising:
a detection device for detecting an electromyographic signal of the person;
a breathing influencing device; and
a control device for controlling the detection device and the breathing influencing device, the control device being configured to determine information on a muscle state of an inspiratory muscle of the person based on the electromyographic signal, and to operate the breathing influencing device as a function of the information on the muscle state in a training mode, which is limited in time, with a training intensity, wherein the control device is further configured to operate as a function of the information on the muscle state at a first time in a normal mode with a first training intensity and at a second time in a training mode with a second training intensity, and wherein the control device is further configured to adapt the training mode and the second training intensity during the normal mode of the breathing influencing device as a function of the information on the muscle state for future second times.

20. Breathing unit for ventilating a person in accordance with claim 19, wherein the control device is further configured to carry out the training mode as a change in a breathing assist of the person, which change in limited in time, for training the inspiratory muscles of the person.

21. A process comprising the steps of:
providing a program code, wherein, when the program code is executed on a computer, on a processor or on a programmable hardware component;
providing a breathing influencing device comprising: a detection device for detecting an electromyographic signal of the person; a breathing influencing device; and a control device for controlling the detection device and the breathing influencing device, the control device being configured to determine information on a muscle state of an inspiratory muscle of the person based on the electromyographic signal, and to operate the breathing influencing device as a function of the information on the muscle state in a training mode, which is limited in time, with a training intensity;
with the execution of the program code detecting an electromyographic signal of a person;
with the execution of the program code generating information on a muscle state of an inspiratory muscle of the person based on the electromyographic signal; and
with the execution of the program code controlling the breathing influencing device as a function of the information on the muscle state in the training mode, which is limited in time.
